SEQUENCE LISTING (8 sequences)

SEQ ID NO: 1 - protein, 199 aa, Norovirus, genogroup II, genotype 4
MKMASNDASPSDGSTANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKVIFAAVPPNFPTEGLSPSQVTMFPHIIVDVRQLEPVLIPLPDVRNNFYHYNQSNDSTIKLIAMLYTPLRANNAGDDVFTVS

SEQ ID NO: 2 - protein, 226 aa, Influenza A virus H7N9 subtype
CSKGKRTVDLGQCGLLGTITGPPQCDQFLEFSADLIIERREGSDVCYPGKFVNEEALRQILRESGGIDKETMGFTYNGIRTNGVTSACKRSGSSFYAEMKWLLSNTDNAAFPQMTKSYKNTRKSPAIIVWGIHHSVSTAEQTKLYGSGNKLVTVGSSNYQQSFVPSPGARPQVNGLSGRIDFHWLILNPNDTVTFSFNGAFIAPDRASFLRGKSMGIQSGVQVDAN

SEQ ID NO: 3 - protein, 233 aa, Influenza A virus H1N1 subtype
CKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHD

SEQ ID NO: 4 - protein, 225 aa, Influenza A virus H3N2 subtype
CDSPHQILDGENCTLIDALLGDPQCDGFQNKKWDLFVERSKAYSNCYPYDVPDYASLRSLVASSGTLEFNNESFNWTGVTQNGTSSACKRKSNNSFFSRLNWLTHLKFKYPALNVTMPNNEKFDKLYIWGVHHPGTDNDQIFLYAQASGRITVSTKRSQQTVIPNIGSRPRVRNIPSRISIYWTIVKPGDILLINSTGNLIAPRGYFKIRSGKSSIMRSDAPIGK

SEQ ID NO: 5 - protein, 237 aa, Influenza B virus Yamagata stain
CPNCLNCTDLDVALGRPMCVGTTPSAKASILHEVRPVTSGCFPIMHDRTKIRQLPNLLRGYENIRLSTQNVINAEKAPGGPYRLGTSGSCPNATSRSGFFATMAWAVPGDNNKTATGPLTVEVPYICTKGEDQITVWGFHSDSKTRMRSLYGDSNPQKFTSSANGVTTHYVSQIGGFPDQTEDGGLPQSGRIVVDYMVQKPGKTGTIVYQRGVLLPQKVWCASGRSKVIKGSLPLIG

SEQ ID NO: 6 - protein, 4 aa, Artificial Sequence
  HHHH Linker
HHHH

SEQ ID NO: 7 - protein, 4 aa, Artificial Sequence
  GGGG Linker
GGGG

SEQ ID NO: 8 - protein, 4 aa, Artificial Sequence
  GSGS Linker
GSGS

(19) United States
(12) Patent Application Publication
(10) Pub. No.: US 2024/0131145 A1
(43) Pub. Date: Apr. 25, 2024

(54) INFLUENZA VACCINE COMPOSITIONS AND METHODS OF USING SAME

(71) Applicant: Children's Hospital Medical Center, Cincinnati, OH (US)

(72) Inventors: Ming TAN, Cincinnati, OH (US); Xi Jason JIANG, Cincinnati, OH (US)

(21) Appl. No.: 18/276,905

(22) PCT Filed: Feb. 16, 2022
(86) PCT No.: PCT/US22/16535

§ 371 Date: Aug. 10, 2023

Related U.S. Application Data
(60) Provisional application No. 63/162,369, filed on Mar. 17, 2021.
(60) Provisional application No. 63/149,742, filed on Feb. 16, 2021.

(51) Int. Cl. A61K 39/145; A61P 31/16; C07K 14/005; C12N 7/00

(52) U.S. Cl. CPC A61K 39/145; A61P 31/16; C07K 14/005; C12N 7/00; A61K 2039/545; C12N 2760/16122; C12N 2760/16123; C12N 2760/16134; C12N 2760/16222; C12N 2760/16223; C12N 2760/16234; C12N 2770/16022; C12N 2770/16023; C12N 2770/16042

(57) ABSTRACT

The instant disclosure relates to pseudovirus nanoparticles (PVNPs) and compositions comprising PVNPs. The disclosed PVNPs may be comprised of fusion proteins that form an icosahedral structure and a nanoparticle shell. The disclosed fusion proteins may comprise a modified norovirus (NoV) S domain protein; a hemagglutinin I (HA1) antigen of the influenza hemagglutinin I (HA1) of influenza vims; and a peptide linker connecting the C-terminus of the NoV S domain to the HA1 antigen. The modified NoV S domain proteins form the interior nanoparticle shell of said PVNP composition and display the 60 HA1 antigens on the surface of the nanoparticle shell. Methods of making and using the PVNPs and compositions containing PVNPs are also disclosed.

### CROSS-REFERENCE TO RELATED APPLICATIONS

This application claims the benefit of, and priority to, U.S. Provisional Application No. 63/162,369, filed Mar. 17, 2021 and U.S. Provisional Application No. 63/149,742, filed Feb. 16, 2021, the contents of each are incorporated by reference in their entirety for all purposes.

### STATEMENT REGARDING FEDERALLY-SPONSORED RESEARCH

This invention was made with government support under AI 148426 awarded by the National Institutes of Health. The government has certain rights in the invention.

### REFERENCE TO SEQUENCE LISTING

A Sequence Listing submitted as an ASCII text file via EFS-Web is hereby incorporated by reference in accordance with 35 U.S.C. § 1.52e. The name of the ASCII text file for the Sequence Listing is SequenceListingCHMC_ST25.txt, the date of creation of the ASCII text file is Feb. 15, 2022, and the size of the ASCII text file is 11 KB.

### BACKGROUND

Influenza viruses (IVs), members of the family Orthomyxoviridae, are single-stranded, negative-sense RNA viruses causing deadly, epidemic influenza (flu) disease in both humans and animals. WHO and other organizations estimated that seasonal flu leads to 250,000-500,000 deaths per annum globally despite of applications of the current flu vaccines [1-3]. Even with implementation of current influenza vaccines, influenza still claims up to 500,000 lives worldwide annually, indicating a need for a better vaccine strategy. Thus, flu continues to be a significant global health burden, and new flu control and prevention strategies with improved efficacy are urgently needed.

Further, given the need for improved understanding of influenza viruses, improved research tools, such as reagents for the study of influenza virus, are likewise needed. The instant disclosure seeks to address one or more of the aforementioned needs in the art.

### BRIEF SUMMARY

Disclosed herein are vaccine compositions, in particular, influenza vaccine compositions, or “pseudovaccine nanoparticles” (PVNPs), having an HA1 antigen presented using a norovirus (NoV) S domain platform. The vaccine composition may comprise a platform formed from a recombinant fusion protein comprising a norovirus (NoV) S domain protein, a linker protein domain connected to the norovirus S domain protein, and an influenza antigen sequence connected via a linker. Further disclosed are methods of using and making PVNPs and compositions containing PVNPs.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Production and characterization of His-tagged S-HA1 PVNPs. (a) Schematic construct of the His-tagged S-HA1 proteins. S, modified norovirus S domain; hinge, the hinge region of norovirus VP1; HA1 the HA1 antigen of H7 subtype; His, His tag. (b) SDS-PAGE of the purified S-HA1 protein (arrow) at ˜52 kDa. Lanes 1/2/3, purified proteins of three preparations; M, protein standards with indicated molecular sizes. (c) An elution curve of gel-filtration chromatography of the purified S-HA1 protein through a size exclusion column. The major PVNP elution peak and a minimal peak of S-HA1 protein monomers are indicated. The two elution peaks were calibrated using the S60-VP8* nanoparticles (˜3.4 mDa) and GST dimers (52 kDa) with their elution positions shown by star symbols on a red dashed line. Y-axis shows UV (A280) absorbances (mAU), while X-axis indicates elusion volume (ml). (d) A representative micrograph of transmission electronic microscopy (TEM) showing the S-HA1 PVNPs.

FIG. 2 Generation and characterization of tag-free S-HA1 PVNPs. (a) Schematic construct of the tag-free S-HA1 protein. S, modified norovirus S domain; hinge, the hinge region of norovirus VP1; HA1 the HA1 antigen. (b) SDS-PAGE of (NH₄)₂SO₄ (0.7 and 1.2 M) precipitated tag-free S-HA1 proteins (arrow). M, protein standards with indicated molecular sizes. (c) A gel-filtration elution curve of the (NH₄)₂SO₄-purified S-HA1 protein through a size exclusion column. The major PVNP elution peak and the minimal S-HA1 monomers peak (arrow) are shown. The elution peaks were calibrated using the S60-VP8* nanoparticles (˜3.4 mDa) and GST dimers (52 kDa) with their elution positions shown by star symbols on a red dashed line. Y-axis shows UV (A280) absorbances (mAU), while X-axis indicates elusion volume (mL). (d) SDS PAGE of the major peaks of the gel-filtration (GF) and ion exchange (IE) chromatography. M, protein standards. (e) An anion exchange elution curve of the tag-free S-HA1 protein. X- and Y-axes are explained as in (c). The red dashed line shows the linear gradient increase of the elution buffer B (0%-100%) with a star symbol showing the percentage of the elution buffer B corresponding to the elution of the PVNPs. The major PVNP elution peak is indicated. (f) and (g) CsCl density gradient centrifugation of the tag-free S-HA1 protein. (f) A photo of the CsCl density gradient after centrifugation with a visible protein band (arrow). (g) The gradient was fractionated into 21 portions showing their relative locations compared with (f). The relative S-HA1 protein concentrations of the fractions were determined by EIA. Y-axis shows the fraction numbers, while X-axis indicates signal intensities (OD) with a red dashed line showing the cut-off signal at OD=0.1. (h) A TEM micrograph of the S-HA1 protein of fraction 15 showing the tag-free S-HA1 PVNPs.

FIG. 3 Structures of the His-tagged, 21-nm S60-HA1 PVNPs. (a) and (b) The 3D structures of the 21-nm S60-HA1 PVNP (a) and its S60 nanoparticle core (b) reconstructed by cryoEM method, revealing a T=1 icosahedral symmetry. The images are shown at five-fold axes. The colored bar in between indicates the radii of the structures. A surface HA1 trimer is indicated by a yellow dashed triangle. (c) Fitting of the crystal structure of the inner shell of the 60 valent feline calicivirus capsid (PDB ID: 4PB6, orange, cartoon representation) into the cryoEM density map of the S60 nanoparticle region. (d) Fitting of the crystal structures of HA1 trimers of an H7N9 flu virus (PDB ID: 4LN3, cyan, cartoon representation) into the cryoEM density maps of the surface HA1 trimer regions with one HA1 trimer shown in red. (e) and (f) The structural model of the S60-HA1 PVNP in PDB format resulted from the above fitting showing a transparent front (e) and a transparent cut (f) views at five-fold axes. The inner S60 nanoparticle shell is shown in orange cartoon representation, while the HA1 antigens are shown in transparent surface model with cartoon representation inside (cyan). (g) Structure of the S60-HA1 PVNP in two color schemes, showing the S60 nanoparticle core (orange) and the 60 surface-displayed HA1 antigens (cyan), forming 20 HA1 trimers. (h) An S60 nanoparticle model (orange) based on the crystal structure of the T=1 VLP of feline calicivirus (PDB ID: 4PB6), showing the 60 exposed C-termini (green) of the S domain in triangle patterns (shown by a yellow dashed triangle). The corresponding HA1 trimers are shown in the 560-HA1 images in (a), (c), (d), and (g).

FIG. 4. Structures of the His-tagged, 16-nm 560-HA1 PVNPs. (a) and (b) The 3D structures of the 16-nm S60-HA1 PVNP (a) and its S60 nanoparticle core region (b) reconstructed by cryoEM method, revealing a T=1 icosahedral symmetry. The images are shown at five-fold axes. The colored bar in between indicates the radii of the structures. A surface HA1 trimer is indicated by a dashed triangle. (c) The cutting view of a PVNP showing its central structure. (d) Fitting of the crystal structure of an H7 HA1 trimer (PDB ID: 4LN3, red, cartoon representation) into the cryoEM density map of a transparent HA1 trimer region. (e) Structure of the 16-nm 560-HA1 PVNP in two color schemes, showing the S60 nanoparticle core (orange) and the 60 surface-displayed HA1 antigens (cyan), forming 20 HA1 trimers. (f) An S60 nanoparticle model (orange) based on the crystal structure of the T=1 VLP of feline calicivirus (PDB ID: 4PB6), showing the 60 exposed C-termini (green) of the S domain in triangle patterns (yellow dashed triangle). The corresponding HA1 trimers are shown in the 560-HA1 images in (a), (b), (d), and (e).

FIG. 5 Structures of the tag-free, 21-nm 560-HA1 PVNPs. (a) and (b) The 3D structures of the 21-nm 560-HA1 PVNP (a) and its S60 nanoparticle core region (b) reconstructed by cryoEM method, revealing a T=1 icosahedral symmetry. The images are shown at five-fold axes. The colored bar in between indicates the radii of the structures. A surface HA1 trimer is indicated by a yellow dashed triangle. (c) Fitting of the crystal structure of the inner shell of the 60-valent feline calicivirus capsid (PDB ID: 4PB6, orange, cartoon representation) into the cryoEM density map of the S60 nanoparticle region. (d) Fitting of the crystal structure of the H7 HA1 trimer (PDB ID: 4LN3, red, cartoon representation) into the cryoEM density map of a surface HA1 trimer regions. (e) Structure of the 560-HA1 PVNP in two color schemes, showing the S60 nanoparticle core (orange) and the 60 surface-displayed HA1 antigens (cyan), forming 20 HA1 trimers. (f) An S60 nanoparticle model (orange) based on the crystal structure of the T=1 VLP of feline calicivirus (PDB ID: 4PB6), showing the 60 exposed C-termini (green) of the S domain in triangle patterns (yellow dashed triangle). The corresponding HA1 trimers are shown in the 560-HA1 images in (a), (c), (d), and (e).

FIG. 6. Antigenic reactivity and receptor binding function of the 560-HA1 PVNPs. (a) Antigenic reactivity of the 560-HA1 PVNPs to H7 HA-specific antibody in EIA assays. Y-axis indicates EIA signals in optical density (OD), while X-axis shows the serially diluted PVNPs at indicated concentrations before (blue) and after (red) boiling treatment, using the S60 nanoparticles (yellow) as controls. (b) Sialoglycans binding specificity of the 560-HA1 PVNPs in EIAs. Y-axis indicates binding signals (OD), while X-axis shows various glycans with specific 2,3-, 2,6-, or 2,8-linked sialic acids. The positively binding glycans are indicated by star symbols. The S60 nanoparticle is used as negative controls. The cutoff signal value (OD=0.1) is shown by a dashed line in both (a) and (b). (c) Hemagglutination of human erythrocytes by the 560-HA1 PVNPs. The protein concentrations of the serially diluted 560-HA1 PVNPs and the S60 nanoparticles are shown at the bottom of the panel. The S60 nanoparticles and PBS are used as negative controls.

FIG. 7. Immune responses of the 560-HA1 PVNPs in mice. (a) HA1-specific IgG titers elicited by the polyvalent 560-HA1 PVNPs (S60-HA1) and the dimeric GST-HA1 fusion protein. (b) and (c) Hemagglutination inhibition (HI) titers of the PVNP- and GST-HA 1-immunized mouse sera against hemagglutinations of the S60-HA1 PVNPs (b) and commercial recombinant hemagglutinin (HA) proteins of H7N9, H3N2, and H1N1 subtypes (Sino Biological) (c). Y-axes indicate IgG titers (a) or HI titers (b) and (c), while X-axes indicate various immunogens (a), various immunogen-immunized mouse sera (b), or HA proteins of various subtypes used for the HI assays (c). The S60 nanoparticles in (a) and the S60 nanoparticle-immunized mouse sera (b) (both labeled as S60) are negative controls. The control in (c) is the result of HI assay using the S60 nanoparticle-immunized mouse sera and the recombinant H7 HA protein. Statistic differences between data groups are shown as ** for highly significant with P-values<0.01, and *** for extremely significant with P-values<0.001.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children.

“Sequence identity” as used herein indicates a nucleic acid sequence that has the same nucleic acid sequence as a reference sequence or has a specified percentage of nucleotides that are the same at the corresponding location within a reference sequence when the two sequences are optimally aligned. For example, a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, such as at least 10, 11, 12, 13, 14, 15, 16, 17, 18, 19, 20, 21, 22, 23, 24, or 25 contiguous nucleotides, or the full-length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications. In one aspect, a codon optimized sequence may share less than 95% sequence identity, less than 90% sequence identity, less than 85% sequence identity, less than 80% sequence identity, or less than 75% sequence identity to a naturally occurring or wild-type sequence (e.g., a naturally occurring or wild-type mRNA sequence encoding a polypeptide or protein of interest (e.g., an antigenic protein or antigenic polypeptide)). In one aspect, a codon-optimized sequence may share between 65% and 85% (e.g., between about 67% and about 85%, or between about 67% and about 80%) sequence identity to a naturally occurring sequence or a wild-type sequence (e.g., a naturally occurring or wild-type mRNA sequence encoding a polypeptide or protein of interest (e.g., an antigenic protein or polypeptide)). In some aspects, a codon-optimized sequence may share between 65% and 75%, or about 80% sequence identity to a naturally occurring sequence or wild-type sequence (e.g., a naturally occurring or wild-type mRNA sequence encoding a polypeptide or protein of interest (e.g., an antigenic protein or polypeptide)).

Structurally, influenza virus is covered by a lipid envelope, two surface proteins, known as hemagglutinin (HA) and neuraminidase (NA), respectively, which protrude outward from the envelope, forming the viral spikes. They are numbered distinctly to name various influenza A virus (IAV) subtypes through different HA-NA combinations. The HA and NA play key roles in IV replication cycles, and thus are believed to be excellent targets of vaccines and antiviral drugs.

Disclosed are unique 560-HA1 pseudovirus nanoparticles (PVNPs) that may display the receptor-binding HA1 domains of influenza viruses. In one aspect, each self-assembled 560-HA1 PVNP may comprise a T=1 icosahedral S60 nanoparticle that resembles the inner shell of norovirus capsid and 60 surface-displayed HA1 antigens that are excellent vaccine targets. Soluble 560-HA1 PVNPs presenting HA1 antigens of H7N9 influenza virus subtypes have been produced efficiently in large amount. Their three-dimensional (3D) structures have been solved by cryogenic electron microscopy. The PVNP-displayed HA1 antigens react with HA-specific antibody, and retain authentic sialic acid binding specificity and hemagglutinate human erythrocytes. The PVNPs are highly immunogenic, eliciting high titers of HA1-specific antibodies in mice and the mouse sera strongly inhibited hemagglutinations of homologous and heterologous influenza virus HA proteins. Therefore, the 560-HA1 PVNPs may be used as reagents to study influenza viruses, as well as a vaccine tactic against influenza disease.

In one aspect, a pseudovirus nanoparticle (PVNP) composition is disclosed. The PVNP may comprise an aggregate of fusion proteins, the fusion proteins forming an icosahedral structure. The nanoparticle shell of the structure may be comprised of the fusion protein, the fusion protein comprising a modified norovirus (NoV) S domain protein, a hemagglutinin I (HA1) antigen of the influenza hemagglutinin I (HA1) of influenza virus, and a peptide linker. The peptide linker may connect the C-terminus of said NoV S domain to said HA1 antigen. The modified NoV S domain protein forms the interior nanoparticle shell of said PVNP composition. The PVNP formed from the fusion protein may have T=1 icosahedral symmetry. In this aspect, the inner nanoparticle shell may display 60 exposed C-termini of said S domain in a triangular pattern, 60 HA1 antigens being displayed on the surface of said nanoparticle shell, wherein the 60 HA1 antigens form 20 HA1 trimers. In one aspect, the PVNP may have a diameter of about 20 to about 21 nanometers, or about 21 nanometers. In one aspect, the HA1 antigen formed on the PVNP particle may be glycosylated. In one aspect, the fusion protein used to form the PVNP may be tag-free.

NoV S Domain. The Norovirus (NoV) S domain sequence used in the fusion protein and subsequent PVNPs may be GenBank AC #: AY038600.3, residue 1 to 221. This sequence may contain a mutation, in particular, a change to Arginine (R) at position 69 to an alanine (A). This mutation destroys the exposed protease cleavage site and subsequently stabilizes the S domain protein. This sequence, containing the R69A is referred to as the “modified S domain” or “569A protein.” It may be understood that additional mutations to the position 69 may be used to render inoperable the protease cleavage site. The NoV S domain may comprise at least 90%, or at least 91%, or at least 92% or at least 93%, or at least 94%, or at least 95% or at least 96%, or at least 97%, or at least 98%, or at least 99%, or 100% homology to the sequence MKMASNDASPSDGSTANLVPEVNNEVMALEPVVGAAIAAPVAGQQNVIDPWIRNNF VQAPGGEFTVSPANAPGEILWSAPLGPDLNPYLSHLARMYNGYAGGFEVQVILAGN AFTAGKVIFAAVPPNFPTEGLSPSQVTMFPHIIVDVRQLEPVLIPLPDVRNNFYHYNQS NDSTIKLIAMLYTPLRANNAGDDVFTVS (SEQ ID NO: 1). In one aspect, the PVNP sequence may be wildtype at one, two, three, or all four positions V57, Q58, or 5136, and M140. In one aspect, the NoV S domain sequence may be wildtype at all positions V57, Q58, or 5136, and M140.

HA1 Antigen. In one aspect, the HA1 antigen comprises a region covering the receptor binding site (RBS) of an influenza virus. In one aspect, the HA1 antigen may be that of an H7N9 influenza virus subtype. For example, the HA1 antigen may be an H7N9 influenza virus subtype having at least 90% sequence homology, or at least 91% sequence homology, or at least 92% sequence homology, or at least 93% sequence homology, or at least 94% sequence homology, or at least 95% sequence homology, or at least 96% sequence homology, or at least 97% sequence homology, or at least 98% sequence homology, or at least 99% sequence homology, or at least 100% sequence homology to CSKGKRTVDLGQCGLLGTITGPPQCDQFLEFSADLIIERREGSDVCYPGKFVNEEALR QILRESGGIDKETMGFTYNGIRTNGVTSACKRSGSSFYAEMKWLLSNTDNAAFPQMT KSYKNTRKSPAIIVWGIHHSVSTAEQTKLYGSGNKLVTVGSSNYQQSFVPSPGARPQ VNGLSGRIDFHWLILNPNDTVTFSFNGAFIAPDRASFLRGKSMGIQSGVQVDAN (SEQ ID NO: 2), HA1 antigen, H7N9 influenza A virus H7N9 subtype (A/Jiangsu/TM261/2017/H7N9, GenBank AC #: AUN86892.1), wherein said HA antigen has a length of from about 200 to about 250 amino acids, or from about 205 to about 245 amino acids, or from about 210 to about 240 amino acids, or from about 215 to about 235 amino acids, or from about 220 to about 230 amino acids, or from about 225 to about 228 amino acids, or 226 amino acids. In one aspect, the HA1 antigen may be that of an of an H1N1 subtype having at least 90% sequence homology, or at least 91% sequence homology, or at least 92% sequence homology, or at least 93% sequence homology, or at least 94% sequence homology, or at least 95% sequence homology, or at least 96% sequence homology, or at least 97% sequence homology, or at least 98% sequence homology, or at least 99% sequence homology, or at least 100% sequence homology to CKLRGVAPLHLGKCNIAGWILGNPECESLSTASSWSYIVETPSSDNGTCYPGDFIDYE ELREQLSSVSSFERFEIFPKTSSWPNHDSNKGVTAACPHAGAKSFYKNLIWLVKKGNS YPKLSKSYINDKGKEVLVLWGIHHPSTSADQQSLYQNADTYVFVGSSRYSKKFKPEI AIRPKVRDQEGRMNYYWTLVEPGDKITFEATGNLVVPRYAFAMERNAGSGIIISDTP VHD (SEQ ID NO: 3), HA1 antigen, H1N1 influenza A virus H1N1 subtype (A/California/04/2009/H1N1, GenBank AC #: ACP41105.1), wherein said HA antigen has a length of from about 200 to about 250 amino acids, or from about 205 to about 245 amino acids, or from about 210 to about 240 amino acids, or from about 215 to about 235 amino acids, or from about 220 to about 235 amino acids, or from about 225 to about 235 amino acids, or 233 amino acids. In one aspect, the HA1 antigen may be that of an of an H3N2 subtype having at least 90% sequence homology, or at least 91% sequence homology, or at least 92% sequence homology, or at least 93% sequence homology, or at least 94% sequence homology, or at least 95% sequence homology, or at least 96% sequence homology, or at least 97% sequence homology, or at least 98% sequence homology, or at least 99% sequence homology, or at least 100% sequence homology to CDSPHQILDGENCTLIDALLGDPQCDGFQNKKWDLFVERSKAYSNCYPYDVPDYAS LRSLVASSGTLEFNNESFNWTGVTQNGTSSACKRKSNNSFFSRLNWLTHLKFKYPAL NVTMPNNEKFDKLYIWGVHHPGTDNDQIFLYAQASGRITVSTKRSQQTVIPNIGSRPR VRNIPSRISIYWTIVKPGDILLINSTGNLIAPRGYFKIRSGKSSIMRSDAPIGK (SEQ ID NO: 4), HA1 antigen, H3N2 influenza A virus H3N2 subtype (A/Western Australia/69/2005/H3N2, GenBank AC #: ABJ53460.1), wherein said HA antigen has a length of from about 200 to about 250 amino acids, or from about 205 to about 245 amino acids, or from about 210 to about 240 amino acids, or from about 215 to about 235 amino acids, or from about 220 to about 230 amino acids, or from about 225 to about 228 amino acids, or 225 amino acids. In one aspect, the HA1 antigen may be that of an influenza B virus subtype having at least 90% sequence homology, or at least 91% sequence homology, or at least 92% sequence homology, or at least 93% sequence homology, or at least 94% sequence homology, or at least 95% sequence homology, or at least 96% sequence homology, or at least 97% sequence homology, or at least 98% sequence homology, or at least 99% sequence homology, or at least 100% sequence homology to CPNCLNCTDLDVALGRPMCVGTTPSAKASILHEVRPVTSGCFPIMHDRTKIRQLPNLL RGYENIRLSTQNVINAEKAPGGPYRLGTSGSCPNATSRSGFFATMAWAVPGDNNKT ATGPLTVEVPYICTKGEDQITVWGFHSDSKTRMRSLYGDSNPQKFTSSANGVTTHYV SQIGGFPDQTEDGGLPQSGRIVVDYMVQKPGKTGTIVYQRGVLLPQKVWCASGRSK VIKGSLPLIG (SEQ ID NO: 5), HA1 antigen, influenza B virus subtype Yamagata stain (B/Yamagata/16/88, GenBank AC #: AAD02807.1), wherein said HA antigen has a length of from about 200 to about 250 amino acids, or from about 205 to about 245 amino acids, or from about 210 to about 240 amino acids, or from about 215 to about 240 amino acids, or from about 225 to about 245 amino acids, or from about 230 to about 240 amino acids, or 237 amino acids.

In one aspect, the HA Antigen sequence comprises from about 200 to about 300 amino acids. In one aspect, the HA Antigen sequence comprises from about 215 to about 275 amino acids. In one aspect, the HA Antigen sequence comprises from about 220 to about 250 amino acids. In one aspect, the HA Antigen sequence comprises from about 225 to about 240 amino acids.

Linker. In one aspect, the fusion protein may comprise an amino acid linker component. In one aspect, the linker may comprise a sequence selected from HHHH (SEQ ID NO: 6), GGGG (SEQ ID NO:7), and GSGS (SEQ ID NO:8).

In one aspect, the pseudovirus nanoparticle (PVNP) may comprise a plurality of fusion proteins, said fusion protein comprising a modified norovirus (NoV) S domain protein, a hemagglutinin I (HA1) antigen of the influenza hemagglutinin I (HA1) of an influenza virus, said S domain protein and HA1 antigen being connected via a peptide linker, wherein the plurality of fusion proteins forms a nanoparticle having an icosahedral structure, wherein the nanoparticle has 60 HA1 antigens presented at the surface of the nanoparticle, the 60 HA1 antigens arranged to form 20 HA trimers, and wherein the HA1 antigen has a sequence length of from about 225 to about 240 amino acids, and from about 95% to 100% sequence homology to a sequence selected from SEQ NO: 2, SEQ NO: 3, SEQ NO: 4 or SEQ NO: 5.

In one aspect, the disclosed PVNPs and compositions comprising PVNPs may be used for the study of influenza antigens. In this aspect, the disclosed PVNPs may be used as a reagent for the study of an influenza virus. For example, the disclosed PVNPs may be used to detect humoral immunity to influenza virus infection in a vertebrate by providing an effective antibody-detecting amount of a disclosed PVNP. The PVNP may be contacted with a sample of bodily fluid from a vertebrate to be examined for influenza virus infection. Influenza virus specific antibodies contained in the sample may be allowed to bind to the PVNP to form an antigen-antibody complex. The complex may then be separated from unbound complexes and contacted with a detectably labeled immunoglobulin-binding agent. The amount of the detectably labeled immunoglobulin-binding agent that is bound to the complexes may then be determined.

In one aspect, the reagent for the study of an influenza virus may comprise any of the aforementioned compositions comprising a PNVP. In one aspect, the reagent may comprise a PNVP capable of remaining structurally intact after storage at −80° C. and −20° C. for at least a year, and at 4° C. for at least six months. In one aspect, the reagent comprising a PNVP may be capable of retaining structural integrity after lyophilization and rehydration treatment. In one aspect, the reagent comprising a PVNP may comprise a polyvalent HA1 antigen, the polyvalent HA1 antigen having one or more features selected from improved antigenic reactivity, improved receptor binding, and improved immune response, as compared to a commercially available recombinant monomeric or trimeric HA proteins, particularly wherein such feature is improved or enhanced as compared to commercially available antigens for such study.

The disclosed compositions may be formulated for administration to an individual in need thereof, according to one or more disclosed methods. For the purposes of administration, the PVNP compositions may comprise any suitable pharmaceutically acceptable carrier, in addition to additional active substances, such as, for example, a therapeutically-active substance, a prophylactically-active substance, or a combination of both. Vaccine compositions may be sterile, pyrogen-free or both sterile and pyrogen-free. Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient (PVNP). By way of example, the composition may comprise between 0.1% and 100%, e.g., between 0.5 and 50%, between 1-30%, between 5-80%, at least 80% (w/w) active ingredient (PVNP).

The PVNP may be formulated in a composition comprising one or more of an adjuvant and a preservative. As used herein the term “adjuvant” refers to a compound that, when used in combination with a specific immunogen (e.g. a VLP) in a formulation, augments or otherwise alters or modifies the resultant immune response. Modification of the immune response includes intensification or broadening the specificity of either or both antibody and cellular immune responses. Modification of the immune response can also mean decreasing or suppressing certain antigen-specific immune responses. Exemplary, adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MF-59, Novasomes®, MHC antigens may also be used.

In one aspect, the disclosed PVNP compositions may be used according to the disclosed methods, more particularly, to induce substantial immunity to influenza virus infection or at least one symptom thereof when administered to a subject. As used herein the term “substantial immunity” refers to an immune response in which when VLPs of the invention are administered to a vertebrate there is an induction of the immune system in said vertebrate which results in the prevention of influenza infection, amelioration of influenza infection or reduction of at least one symptom related to influenza virus infection in said vertebrate.

In one aspect, a method of inducing substantial immunity to influenza virus infection or at least one symptom thereof in a subject is disclosed, the method comprising administering at least one effective dose of one or more PVNP or PVNP-containing composition as disclosed herein.

In one aspect, a method of inducing a substantially protective antibody response to influenza virus infection or at least one symptom thereof in a subject, is disclosed, the method comprising administering at least one effective dose of one or more PVNP or PVNP-containing composition as disclosed herein. As used herein the term “substantially protective antibody response” refers to an immune response mediated by antibodies against an influenza virus, which is exhibited by a vertebrate (e.g., a human), that prevents or ameliorates influenza infection or reduces at least one symptom thereof. VLPs of the invention can stimulate the production of antibodies that, for example, neutralizing antibodies that block influenza viruses from entering cells, blocks replication of said influenza virus by binding to the virus, and/or protect host cells from infection and destruction.

In one aspect, a method of inducing a substantially protective cellular immune response to influenza virus infection or at least one symptom thereof in a subject, is disclosed, the method comprising administering at least one effective dose of one or more PVNP or PVNP-containing composition as disclosed herein.

In one aspect, a method of immunizing an individual against one or both of an H7 IAV or H3N2 or H7N9 avian IAV, is disclosed, the method comprising administering one or more PVNP or PVNP-containing composition as disclosed herein.

In one aspect, a method of eliciting an immune response in a subject against an influenza virus in an individual in need thereof against an influenza virus, is disclosed, the method comprising administering a PVNP or PVNP-containing composition, in an amount effective to produce an antigen-specific immune response in an individual.

The PVNP or composition comprising a PVNP may be administered in the form of a dose. Doses may be administered on a schedule, for example, an initial administration with subsequent booster administrations. For example, a second dose may be administered anywhere from two weeks to one year, or from about 1, about 2, about 3, about 4, about 5 to about 6 months, after the initial administration. A third dose may be administered after the second dose and from about three months to about two years, or even longer, or about 4, about 5, or about 6 months, or about 7 months to about one year after the initial administration. The third dose may be optionally administered when no or low levels of specific immunoglobulins are detected in the serum and/or urine or mucosal secretions of the subject after the second dose.

In one aspect, the effective amount may be a total dose of from about 1 μg to about 500 μg, or from about 10 μg to about 250 μg. In one aspect, the effective amount may be a total dose of from about 25 μg to about 150 μg. In some aspects, the effective amount may be a dose of 25 μg or about 50 μg or about 100 μg or about 150 μg administered to the subject a total of two times. In some aspects, the effective amount may be a dose of 500 μg administered to the subject a total of two times. In some aspects, the dosage of the XXXX is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In one aspect, the dose comprising from about 1 to about 150 μg of PVNP.

In one aspect, the PVNP or PVNP-containing composition, or dose thereof may be administered to an individual via a route selected from one or more of intradermal injection, intramuscular injection, or by intranasal administration.

The disclosed vaccine compositions may be administrated with (or include as part of the composition) other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition.

In one aspect, a method of formulating a vaccine that induces substantial immunity to influenza virus infection or at least one symptom thereof to a subject, is disclosed, the method comprising adding to a formulation an effective dose of one or more PVNP as disclosed herein.

In one aspect, a method of making a pseudovirus nanoparticle (PVNP) is disclosed. In this aspect, the method may comprise providing a plasmid comprising gene sequences corresponding to a modified NoV S domain, a linker, and an HA1 antigen; expressing a gene product of said gene sequences in an E. coli or a eukaryotic cell system; lysing the cells of said cell system expressing the S-HA1 protein; and precipitating said gene product from said lysed cell system with (NH₄)₂SO₄; wherein said sequence is tag-free; and wherein said concentration of (NH₄)₂SO₄ is between about 0.7 and about 1.2 M. In this aspect, the cell system may be selected from a yeast system, a baculovirus/insect cell system, and a mammalian cell system.

Dosage Forms and Formulation

In one aspect, active agents provided herein may be administered in a dosage form selected from intravenous or subcutaneous unit dosage form, oral, parenteral, intravenous, and subcutaneous. Suspensions may be formulated according to methods well known in the art using suitable dispersing or wetting agents and suspending agents. The preparation of acceptable aqueous solutions with suitable pH, isotonicity, stability, and the like, is within the skill in the art. In some aspects, a composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative may be typically from about 0.02% to about 2% based on the total weight of the composition, although larger or smaller amounts may be desirable depending upon the agent selected. Reducing agents, as described above, may be advantageously used to maintain good shelf life of the formulation.

Pharmaceutically acceptable carriers include but are not limited to physiological saline, buffered saline, dextrose, sterile water, glycerol, sterile isotonic aqueous buffer, and combinations thereof. In one aspect, the formulation is sterile, non-particulate and/or non-pyrogenic. The compositions may contain pH buffering agents, gelling or viscosity enhancing additives, preservatives, flavoring agents, colors, and the like, depending upon the route of administration and the preparation desired. Additional components may be used to influence the physical state, solubility, stability, rate of in vivo release, and rate of in vivo clearance, and are thus may be selected according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions. When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Each formulation may be specific to the type of device employed and may involve the use of an appropriate propellant material, in addition to diluents, adjuvants, and/or carriers useful in therapy.

In some aspects, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate compositions containing an active agent (PVNP) as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### Examples

The following non-limiting examples are provided to further illustrate aspects of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific aspects that are disclosed and still obtain a like or similar result without departing from the spirit and scope of the invention.

Vaccines remain the most effective approach to control and prevent (e.g., to reduce the incidence of infection, the likelihood of becoming infected, or the severity of infection due to) flu. However, U.S. Centers for Disease Control and Prevention (CDC) records indicate that the effectiveness of conventional flu vaccines based on egg-grown, inactivated IVs was low, ranging from 10% to 60% [4], explaining the continual flu prevalence with significant mortality and morbidity. Most of the vaccines are produced by growing the corresponding IV strains in fertilized chicken eggs, which often leads to adaptive mutations and thus altered antigenicity of the vaccine IVs after many cycles of propagations in eggs. Such mutations have been shown to be an important factor contributing to the low effectiveness of egg-based flu vaccines [5-8]. For example, the egg-grown contemporary H3N2 vaccine strains used since 2016 were found antigenically mismatched circulating H3N2 strains [9, 10], explaining the low efficacy of H3N2 vaccine during the winter seasons of 2016/2017 (33%) and 2017/2018 (22%) [4, 11]. Accordingly, a study to compare human H3N2 antibody responses elicited by egg-based (Fluzone®), cellbased (Flucelvax®), and recombinant HA-based (Flublok®) flu vaccines during the 2017/2018 winter season showed that the recombinant HA-based flu vaccines exhibited significantly higher neutralizing antibody titers than those induced by the egg- and cellbased flu vaccines [8]. Therefore, the recombinant HA-based flu vaccine with original HA sequences represents an approach for higher protective efficacy than the egg/cell-grown vaccines with adaptative mutations.

The immunodominant HA1 domains constitute the heads of IV HA trimers, interacting with host sialic acid receptors to initiate IV infection and thus are an ideal vaccine target. The commercial use of the Flublok® vaccine has demonstrated the feasibility and usefulness of the recombinant HA-based flu vaccine approach. However, the relatively smaller size and low valence of a recombinant HA trimer may be less immunogenic compared with the polyvalent HA antigens on a virus particle. This problem could be solved through displays of the HA antigens by a polyvalent protein nanoparticle platform, such as a 24-valent nanoparticle [12-14] formed by ferritins that are ubiquitous iron storage proteins, or the 60-valent 153-50 nanoparticle that is one of several computationally designed complexes [16]. Alternatively, the HA antigens may be displayed by lipid nanoparticles [17-19]. The results from these studies showed that the polyvalent platforms enhanced the immune responses of the displayed antigens.

Applicant has developed a T=1 icosahedral S60 nanoparticle, which is self-assembled by 60 norovirus (NoV) capsid shell (S) domains, having a C-termini exposed on the surface in triangle patterns [20]. This 60-valent nanoparticle resembles the inner shell of NoV capsid and has been shown to be a useful platform to display VP8* antigens, the receptor-binding domains of rotaviruses, for improved immunogenicity as a rotavirus vaccine candidate [20, 21]. In this study, the receptor-binding HA1 domains of IV HAs were fused to the NoV S domains to generate S60-HA1 pseudovirus nanoparticles (PVNPs) with 60 exposed HA1 antigens arranged into 20 HA1-trimers, resembling those on IV virions. Further disclosed are methods that may be used to generate the self-assembled S60-HA1 PVNPs displaying HA1 antigens of H7N9 IAV subtype. The PVNP-presented HA1s are recognized by HA specific antibody, retain authentic receptor binding function, and agglutinate human red blood cells (RBCs). The PVNPs are highly immunogenic, eliciting high titers of HA1-specific antibody that inhibited hemagglutinations of HA proteins, supporting the notions that the 560-HA1 PVNPs may be used as reagents for IV study and that they may serve as a flu vaccine candidate.

### 2 Materials and Methods

### 2.1 Plasmids for S-HA1 Fusion Protein Expressions

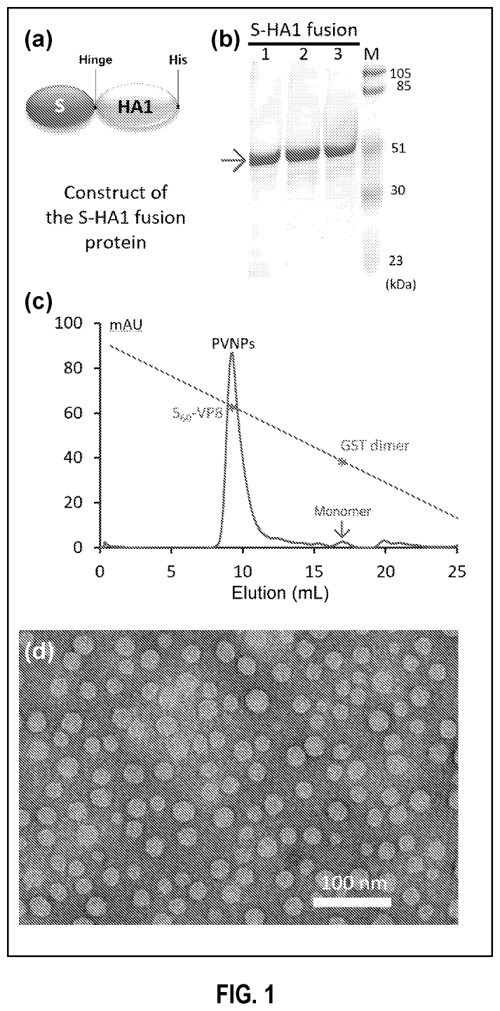

DNA sequences encoding the HA1 antigen of an A/H7N9 strain (GenBank AC #: AUN86892.1, A/Jiangsu/TM261/2017/H7N9, amino acid C60 to N285, 226 aa), were codon-optimized to Escherichia Coli using the codon adaptation tool at http://www.jcat.de and synthesized by GenScript (Piscataway, NJ). The DNA fragment was cloned into the previously made pET-24b (Novagen)-based plasmid that was generated for production of the SR69A-VP8* fusion protein by replacing the VP8*-encoding sequences using the HA1-encoding DNA fragment. R69A refers to the mutation in the NoV S domain to remove the exposed protease site [20]. A linker (HHHH) was added between the S and HA1 domains and a His tag was fused to the C-terminus of HA1 (FIG. 1(a)). A further plasmid without the His-tag encoding sequences was also made for tag-free S-HA1 protein production. As a control for comparisons, a further plasmid for expression of glutathione S-transferase (GST)-HA1 fusion protein were made by cloning the HA1-encoding DNA fragment into the pGEX-4T-1 vector (GE Healthcare Life Sciences) [22]. AUN86892.1 hemagglutinin [Influenza A virus (A/chickenniangsu/TM261/2017(H7N9))], the HA1 fragment, comprises the following sequence:

(SEQ ID NO: 2)
CSKGKRTVDLGQCGLLGTITGPPQCDQFLEFSADLIIERREGSDVCYPG
KFVNEEALRQILRESGGIDKETMGFTYNGIRTNGVTSACKRSGSSFYAE
MKWLLSNTDNAAFPQMTKSYKNTRKSPAIIVWGIHHSVSTAEQTKLYGS
GNKLVTVGSSNYQQSFVPSPGARPQVNGLSGRIDFHWLILNPNDTVTFS
FNGAFIAPDRASFLRGKSMGIQSGVQVDAN.

### 2.2 Tagged Protein Production

Recombinant proteins were expressed using the E. coli (BL21, Arctic) system induced by 0.4 mM isopropyl-β-Dthiogalactopyranoside (IPTG) at 13° C. overnight as described previously [20, 23, 24]. Soluble His-tagged proteins were purified with His-tag binding cobalt resins (Thermo Fisher Scientific, Waltham, MA, USA) according to the instructions of the manufacturers, eluted with 150 mM imidazole (Sigma-Aldrich, St. Louis MO, USA) in 50 mM phosphate buffered saline (PBS, pH 7.2). Soluble GST-tagged proteins were purified using the GST binding resin (Glutathione Sepharose 4B GST-tagged protein purification resin, GE Healthcare Life Sciences) as described previously [24].

### 2.3 Tag-Free Protein Production

The tag-free S-HA1 protein was expressed in E. coli as described above. For purification, clarified bacterial lysis was incubated with ammonium sulfate [(NH₄)₂SO₄] at 1.2 M end concentration for 30 mM to selectively precipitate the S-HA1 proteins as reported previously [25]. Briefly, after centrifugation with an Avanti J26XP centrifuge (Beckman Coulter) using a JA-17 rotor at 10,000 rpm for 20 mM, the protein pellet was collected, washed twice using 1.2 M (NH₄)₂SO₄ solution (in PBS), and then solved in 20 mM tris buffer (pH 7.5). The protein was analyzed and further purified by gel-filtration, ion exchange chromatography, and cesium chloride (CsCl) density gradient centrifugation (see below).

### 2.4 Sodium Dodecyl Sulfate Polyacrylamide Gel Electrophoresis (SDS-PAGE) and Protein Quantitation

Purified proteins were analyzed by SDS-PAGE using 10% separating gels and were quantitated by SDS-PAGE using serially diluted bovine serum albumin (BSA, Bio-Rad) with known concentrations as standards on same gels [26].

### 2.5 Gel Filtration Chromatography

This was performed as described previously [23, 24, 27] using an AKTA Fast Performance Liquid Chromatography system (FPLC, AKTA Pure 25L, GE Healthcare Life Sciences) using a size exclusion column (Superdex 200, 10/300 GL, 25 mL bed volume, GE Healthcare Life Sciences) to analyze the sizes of the S-HA1 proteins as evidence of PVNP formation. The column was calibrated using gel filtration calibration kits (GE Healthcare Life Sciences), while the elution peaks of the PVNPs and S-HA1 monomers were determined using the previously made S60-VP8* nanoparticles (˜3.4 mDa) and GST-dimer (52 kDa). Relative protein concentrations in the effluent were reported as A280 absorbance.

### 2.6 Anion Exchange Chromatography

This was carried out using the same AKTA FPLC system (see above) using a HiPrep Q HP 16/10 column (20 mL bed volume, GE Healthcare Life Sciences) to analyze and purify the tag-free S-HA1 protein. A previous established procedure was modified and applied here. After the column was equilibrated using 7 column volumes (CVs) of 20 mM Tris buffer (pH 8.0, referred as buffer A), protein samples were loaded. The column was washed using 7 CVs of buffer A to remove the unbound proteins, followed by elution of the bound proteins using 7 CVs of buffer B (1 M NaCl in buffer A) via a linear gradient elution manner (0%-100% B). The column was then stripped by 7 CVs of buffer B (100% B), followed by an equilibration using 7 CVs of buffer A. Relative protein concentrations in the effluent were reported as A280 absorbance, while the elution positions of the proteins were shown as percentages of buffer B.

### 2.7 CsCl Density Gradient Ultracentrifugation

This was performed as described previously [20]. Briefly, 0.5 mL of (NH₄)₂SO₄ precipitated S-HA1 protein (see above) was mixed with 10 mL of CsCl solution with a density of 1.3630 g/mL and packed in a 12-mL centrifuge tube. After centrifugation at 288,000×g for 45 h in the Optima L-90K ultracentrifuge (Beckman Coulter), the gradient was fractionated by bottom puncture into 21 fractions (— 0.5 mL each). CsCl densities of fractions were determined according to the refractive index.

### 2.8 Transmission Electron Microscopy (TEM)

TEM was used to inspect the morphology of the PVNPs. After staining with 1% ammonium molybdate, purified S-HA1 proteins were observed under an EM10 C2 microscope (Zeiss, Germany) at 80 kV at magnifications between 15,000× and 30,000× as described elsewhere [20].

### 2.9 Immunization of Mice

Specific pathogen free (SPF) BALB/c mice at ˜6 weeks of age were purchased from the Jackson Laboratory (Bar harbor, ME, USA) and maintained under SPF conditions at the Division of Veterinary Services of Cincinnati Children's Hospital Medical Center (CCHMC). Mice were randomly divided into four groups with 6-8 mice each (n=6-8) and each group was administered with one of following immunogens: (1) 560-HA1 PVNPs (S60-HA1); (2) GST-HA1 fusion protein (GST-HA1); (3) S60 nanoparticle without HA1 antigens as platform control (S60); and (4) PBS as diluent control (PBS) Immunogens were administered with Alum adjuvant (Imject Alum, Thermo Fisher Scientific, USA) at 25 μL/dose (20 μg/mouse/dose). Endotoxin contaminations of the recombinant protein immunogens were removed using Endotoxin Removal Spin Columns (Thermo Scientific™ Pierce, USA). After this treatment, all immunogens for mouse immunization contained less than 1.5 endotoxin units. Mice were immunized intramuscularly (i.m.) in the thigh muscle three times with injection volume of 50 μL at 2-week intervals. Bloods were collected 2 weeks after the final immunization via heart puncture and sera were processed from bloods via an established protocol [26].

### 2.10 Enzyme Immunoassays (EIAs)

EIAs were used to determine HA1 specific antibody titers as described elsewhere [28, 29]. Briefly, commercial recombinant H7 HA protein (Sino Biological) was coated on 96-well plates at 1 μg/mL. After blocked with 5% (w/v) skim milk, plates were incubated with mouse sera at serial dilutions. The bound antibodies were measured by goat-anti-mouse IgG-horse radish peroxidase (HRP) conjugate (1:5,000, MP Biomedicals). IgG titers were defined as maximum dilutions of sera with positive signals. A positive signal was determined by a mean of negative values+3×standard deviation. EIAs were also used to measure S-HA1 PVNPs in the fractions of CsCl density gradient. In this case, the fractions were diluted in 800 folds in PBS and coated on microtiter plates. The PVNPs were detected using in-house made antibody against NoV-like particle (VLP) [30].

### 2.11 Cryogenic Electron-Microscopy (cryoEM)

CryoEM was carried out to reconstruct the three-dimensional (3D) structures of the S-HA1 PVNPs as described previously by Applicant [26, 31, 32]. Briefly, 3 μL of purified PVNPs were dropped onto graphene oxide coated lacey grids (Tedpella #01896), then blotted for 4 s before flashed frozen into ethane using Cp3 cryoplunder (Gatan, Inc., Pleasanton CA, USA) at 90% humidity. Low electron-dose images (˜20 e/Å2) were recorded on Talos F200C cryo-TEM, recorded by Ceta 16M camera at a nominal magnification (57,000×) with a calibrated pixel size of 2.59 Å. The collected micrographs were imported into cryoSPARC/v3 for further processing. A total of >14,000 particles were selected with Gautomatch v0.53 (www.mrc-lmb.cam.ac.uk/kzhang/). Multiple homogeneous class averages were separated using twodimensional (2D) classification in CryoSPARC 3.1 [33]. The His tagged particles were further divided to two classes based on their size difference, among which 6,200 separated particles for the larger, and 5,284 particles for the smaller PVNPs were subjected to ab-initio 3D reconstruction for initial model generation and final homogeneous refinement for further analysis. Similarly, 1,064 separated tag-free PVNPs particles were selected for initial model generation, followed by homogeneous refinement. CryoEM map fitting was conducted using UCSF Chimera software (www.rbvi.ucsf.edu/chimera; version 1.15) using in the crystal structures of the inner shell of the 60-valent feline calicivirus (FCV) VLP (PDB code: 4PB6) and the HA1 heads of H7 HA trimer (PDB code: 4LN3) [35, 36]. The structure of the S60 nanoparticle was modeled using the crystal structure of the 60-valent FCV inner shell (PDB code: 4PB6) using the same UCSF Chimera software.

### 2.12 EIA-Based Glycan Binding Assay

This was used to measure interactions between the S-HA1 PVNPs and sialoglycans. Sialoglycans of (1) Neu5Acα2-8Neu5Acα2-8Neu5Acα-sp-PAA-biotin, (2) Neu5Acβ2,6(Galβ1,3)GalNAcα-PAA-biotin, (3) α-Neu5Ac2,3βGal1,4Glc(3′-sialyllactose)-PAA-biotin, (4) sialyl Lea-PAA-biotin, and (5) sialylLex-PAA-biotin were from GlycoTech (Rockville, MD), while glycans of Neu5Acα2,3Galβ1,3(Fuca1,4)GlcNAcrβ-SpNH-PAA-biotin and Neu5Acα2,3Galβ1,4(Fuca1,3)GlcNAcβ-SpNH-PAA-biotin were from the Consortium for Functional Glycomics (www.functionalglycomics.org/). All glycans were conjugated with polyacrylic acid (PAA) and labeled with biotin. Both PVNPs and the S60 nanoparticle control at 10 μg/mL were coated on microtiter plates (Thermo Scientific) at 4° C. overnight. After blocking with 5% (w/v) nonfat milk, the plates were incubated with the glycans at 2 μg/mL for 60 mM. The bound glycans were detected by streptavidin-HRP conjugates (Jackson ImmunoResearch Laboratories) at 1:5,000 dilution.

### 2.13 EIA-Based Assays of Antigenic Reactivity

This was performed to test the antigenic reactivity of the S-HA1 PVNPs with HA specific antibody as described elsewhere [22]. Briefly, native and boiled S-HA1 PVNPs were serially diluted and coated on microtiter plates (Thermo Scientific™, USA) at 4° C. overnight. After blocking, commercial H7 HA specific antibody (Sino Biological) at 1:1,000 or appropriate dilutions was added to the coated wells. The bound antibody was detected by corresponding HRP-conjugated secondary antibody (Thermo Scientific™, USA) at 1:2,000 dilution.

### 2.14 Hemagglutination and Hemagglutination Inhibition

(HI) assays. The assays were used to measure hemagglutination titers of recombinant HAs and S-HA1 PVNPs using human RBCs that were provided by the Translational Core Laboratory at CCHMC, as described elsewhere [22]. Briefly, 500 μL of 0.5% RBCs in 10 mM PBS were mixed with serially diluted HAs or PVNPs in 96-well V-bottom plates and incubated at 4° C. for 60-100 mM, using the S60 nanoparticles and PBS as negative controls. Hemagglutination titers were defined as the highest dilutions of the HAs or PVNPs that caused hemagglutination. For HI assays, HAs (Sino biological) or PVNPs at 4× hemagglutination titers were incubated with serially diluted serum antibody for 60 mM before they were mixed with RBCs for hemagglutination assays. The HI titers were defined as the maximum serum dilutions that prevented hemagglutination.

### 2.15 Ethics Statement

All animal studies were completed in compliance with the recommendations in the Guide for the Care and Use of Laboratory Animals (23a) of the National Institute of Health (NIH). The protocols were approved by the Institutional Animal Care and Use Committee (IACUC) of the Cincinnati Children's Hospital Research Foundation (Animal Welfare Assurance No. A3108-01).

### 2.16 Statistical Analysis

Statistical differences between two data groups were calculated by GraphPad Prism 9.0 (GraphPad Software, Inc.) using unpaired Student's t-test. Differences were considered non-significant when P-values>0.05 (marked as ns), significant when P-values<0.05 (marked as *), highly significant when P-values<0.01 (marked as **), and extremely significant when P-values<0.001 (marked as ***), respectively.

### 3 Results

### 3.1 Production of His-Tagged S-HA1 PVNPs

S-HA1 fusion protein containing the HA1 antigens of an H7N9 IV (FIG. 1(a)) was produced via the E. coli system. The C terminally His-tagged, soluble S-HA1 protein was purified at yields of ˜20 mg/L bacterial culture using His-tag binding cobalt resin. SDS-PAGE showed the purified protein at an expected size of ˜52 kDa (FIG. 1(b)). Gel-filtration chromatography of the protein revealed a single major elution peak with high molecular size like the previously made S60-VP8* nanoparticles (FIG. 1(c)), while the elution peak corresponding to the S-HA1 protein monomers was minimal, indicating that vast majority of the proteins self-assembled into PVNPs or large complexes. TEM inspection of the major gel-filtration elution peak demonstrated the self-formation of the S-HA1 H7 PVNPs (FIG. 1(d)). Major PVNP population had sizes between 16 and 20 nm with some variations.

### 3.2 Generation of Tag-Free S-HA1 H7 PVNPs

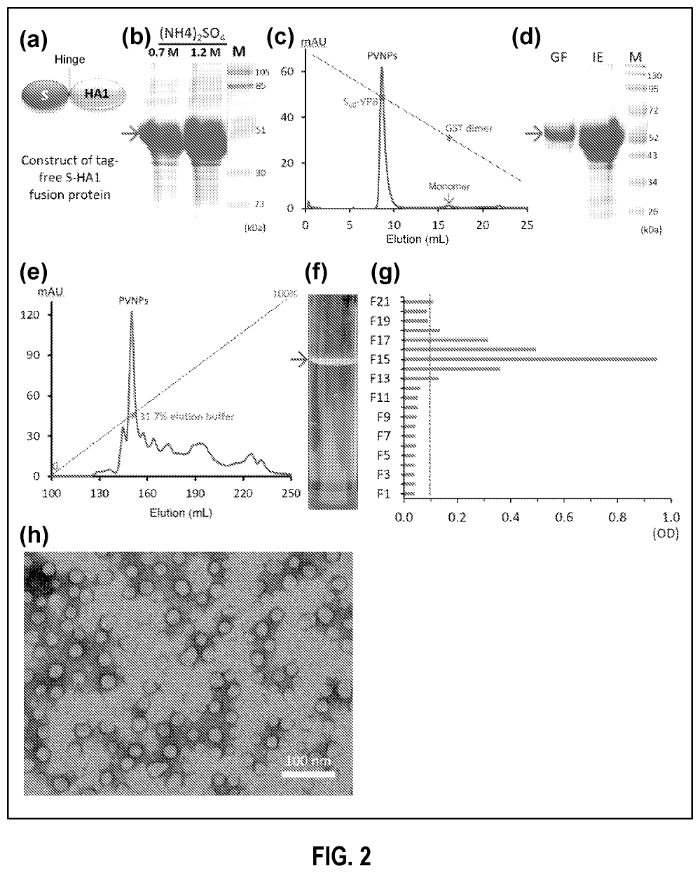

To assess potential impact of the His-tag on PVNP formation and explore alternative production and purification approaches, tag-free S-HA1 protein (FIG. 2(a)) was also produced and purified by selective precipitation using (NH₄)₂SO₄ at concentrations of 0.7 and 1.2 M. This resulted in high protein yields at 30-40 mg/L bacterial culture (FIG. 2(b)). Gel-filtration of the (NH₄)₂SO₄-purified, resolved protein revealed a similar single major elution peak of PVNPs (FIG. 2(c)), confirming that the fusion protein self-assembled into PVNPs. The protein was also analyzed by an anion exchange chromatography (FIG. 2(e)), in which vast majority of the target protein was eluted in the major peak corresponding to 31.7% buffer B, equivalent to an ionic strength of 317 mM NaCl and 20 mM Tris. SDS-PAGE of the major elution peaks of the gel filtration and ion exchange revealed the target proteins at high purity (FIG. 2(d)), supporting that the two chromatography approaches may be used to further purify the (NH₄)₂SO₄₋ precipitated, tag-free S-HA1 proteins. The (NH₄)₂SO₄-purified protein was analyzed further by CsCl density gradient centrifugation, resulting in a visible protein band at a density of ˜1.311 mg/cm3 (FIG. 2(f)). The gradient was fractionated into 21 portions, followed by detection of the PVNPs in the factions by EIA assays using the S domain-specific antibody, confirming the protein band as the S-HA1 protein (FIG. 2(g)). Finally, the protein band was examined by TEM, revealing typical PVNPs (FIG. 2(h)) with better homogeneity than the His-tagged ones (comparing FIG. 2(h) with FIG. 1(d)). These data indicated that, while the His-tags did not substantially affect the PVNP formation, tag-free PVNPs showed better production yields and improved PVNP homogeneity.

### 3.3 Structures of the His-Tagged S-HA1 PVNPs

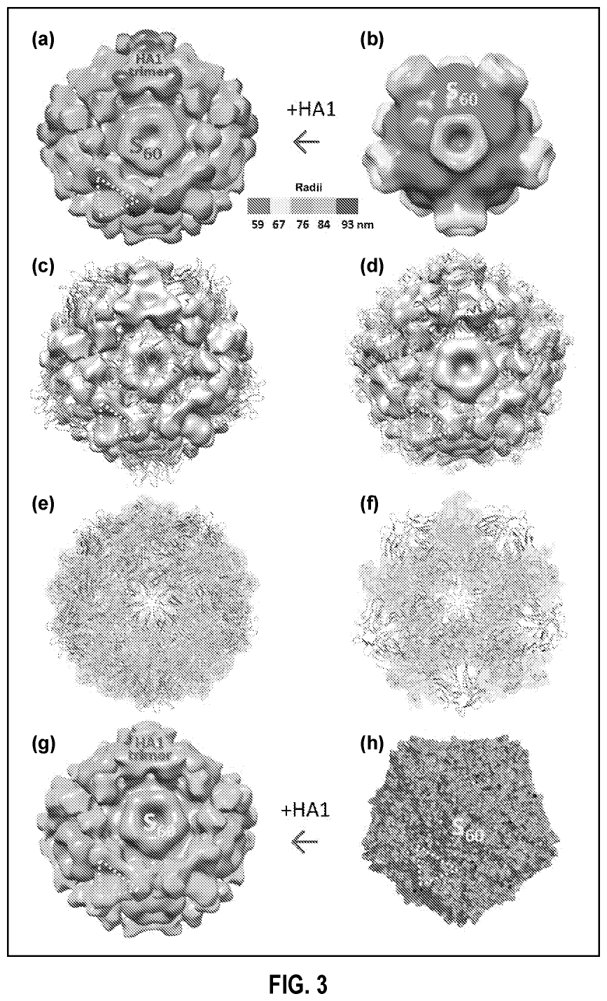

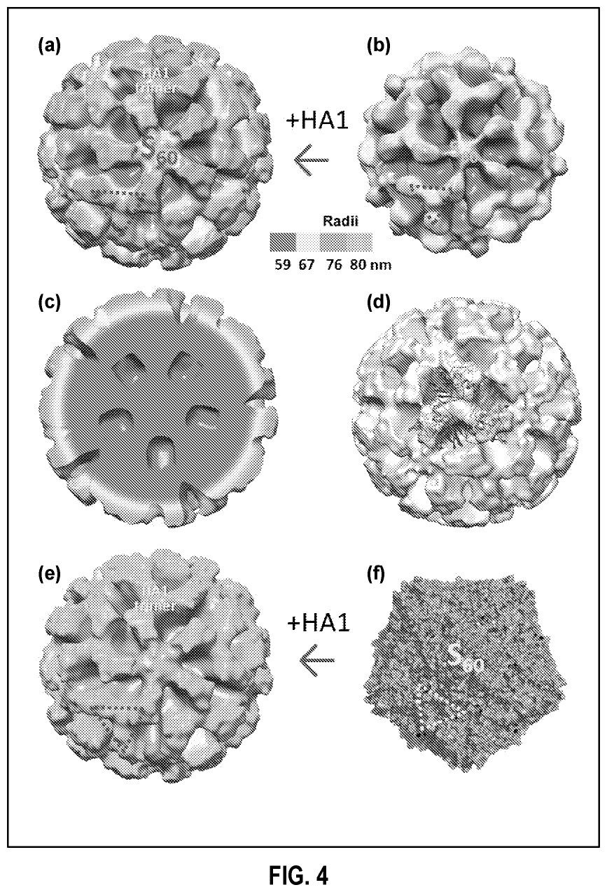

The 3D structures of the S-HA1 PVNPs were reconstructed using cryoEM approach. By focusing on the major populations, Applicant noted and reconstructed two types of the His-tagged PVNPs that have distinct diameters of ˜21 and ˜16 nm, respectively. Both PVNPs were reconstructed at 12-Å resolution (FIGS. 3 and 4) and they are in T=1 icosahedral symmetry, having 60 S-HA1 proteins, thus are named 560-HA1 PVNPs. Fitting of the crystal structure of the S60-nanoparticle (PDB ID: 4PB6) [20, 34] into the cryoEM density map of the 21-nm PVNP inner shell region indicated that their shapes match well, but the PVNP inner shell appears slightly smaller, particularly in the protruding regions at the five-fold axes (FIG. 3(c)). This confirmed the identity of the S60-nanoparticle inner shell of the PVNP (FIG. 3, comparing 3(b) with 3(h)) and revealed that the surface displayed HA1 antigens force the wall of the S60-nanoparticle to go inward structurally, resulting in a compact interior shell. Similar scenario also occurred to the 16-nm, His-tagged PVNP, in which both the wall of the S60-nanoparticle shell and HA1 antigens go further inward, leading to a more compact PVNP with smaller diameter (FIGS. 4(a)-4(c)). The above fitting also showed that the 60 HA1 antigens are located along the sunken grooves among the protrusions at the five-fold axes of the S60 nanoparticle (FIGS. 3(c), 3(g), 4(e)). Noteworthy, the 60 HA1 antigens are arranged into 20 trimers on each PVNP, corresponding to the triangle patterns of the exposed C-termini of the NoV S domains on the S60 nanoparticle (FIGS. 3(g), 3(h), 4(e), 4(f)). These trimer-like HA1s may help to retain their authentic structures and receptor-binding function (see below). When the crystal structures of H7N9 trimers (PDB ID: 4LN3) were fitted into the cryoEM density maps of the trimer-like HA1 regions, they fitted fine despite some discrepancies on the outside edges (FIGS. 3(d) and 4(d)), confirming their structural identities and features. The PVNP structural model in PDB format resulted from the fitting indicated a smaller central lumen than that of the S60-nanoparticle (FIGS. 3(e) and 3(f)). Finally, the two 560-HA1 PVNPs in two color schemes were generated to show the inner shell and the surface trimeric HA1 antigens, respectively (FIGS. 3(g) and 4(e)).

### 3.4 Structures of the Tag-Free S-HA1 PVNPs

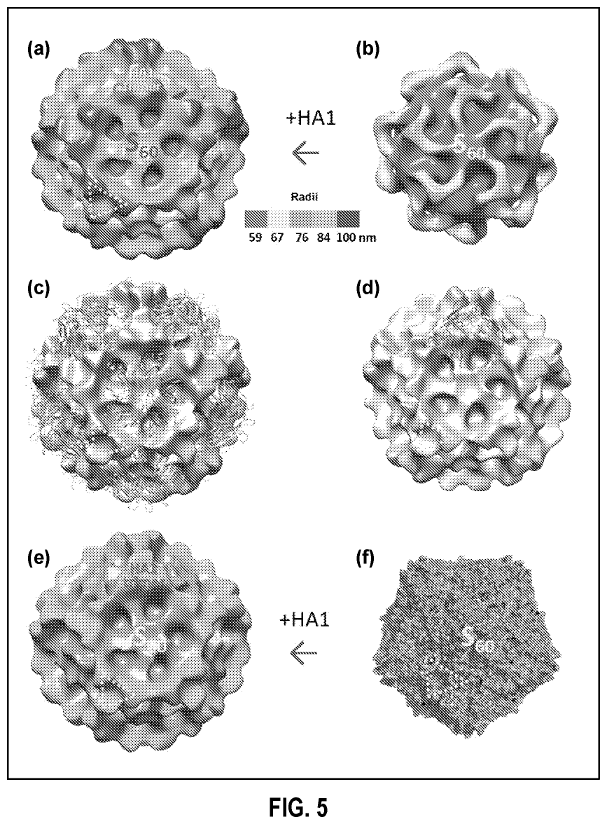

Only the 21-nm PVNP was recognized in the major populations of the tag-free PVNPs. Its 3D structure was solved through cryoEM method at 15-A resolution, revealing the same T=1 icosahedral symmetry (FIG. 5) like the His-tagged PVNPs. Similar organization of the S60 nanoparticle inner shell and 60 exterior, trimer-like HA1 antigens like the His-tagged PVNPs were witnessed by fitting of the 560-nanoparticle and HA1 trimer crystal structures into corresponding regions of the cryoEM density maps, and by structural comparisons between the tag-free and the His-tagged PVNPs (FIGS. 3 and 5). However, differences between the tag-free PVNPs and the tagged ones were also noted. For example, the five-fold axis protrusions of the 21-nm/tag free PVNP go less outward compared with those of the 21-nm/His-tagged PVNP (comparing FIG. 3(b) with FIG. 5(b)). As a result, the grooves among the five-fold axis protrusions of the 21-nm/His-tagged PVNP appear to be deeper than those of the 21-nm/tag-free PVNPs (FIGS. 3(g) and 5(e)). Applicant also noted that the roots of the HA1 antigens of the 21-nm/tag-free PVNP appear to submerge into the S60-nanoparticle wall unlike those of the 21-nm/His-tagged one (comparing FIG. 3(b) with FIG. 5(b)).

### 3.5 Antigenic Reactivity of the PVNPs to HA Antibody

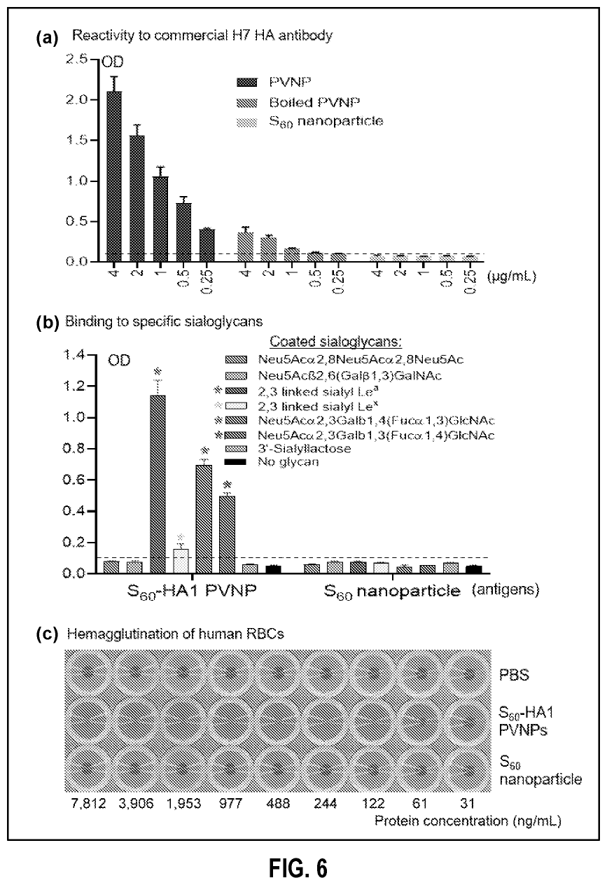

EIA assays showed that the commercial antibody (Sino Biological) specific to IAV H7 HA recognized the His-tagged S60-HA1 PVNPs, as shown by the high and dose-dependent EIA signals (FIG. 6(a)), indicating that the PVNP-displayed HA1s retained their natural conformations. These reactivity signals were mostly gone, when the PVNPs were boiled to destroy conformational epitopes, leaving mainly linear epitopes. As negative controls, the S60 nanoparticles without HA1 antigens did not react with the antibody (FIG. 6(a)). The tag-free PVNPs also revealed similar antigenic reactivity to the H7 HA-specific antibody (data not shown). Due to the similarity of the two PVNP types, downstream experiments were performed using the His-tagged PVNPs by taking advantage of their easy purification with higher purity.

### 3.6 Receptor Binding Function of the PVNP-Displayed HA1s

The sialic acid binding ability and specificity of the S60-HA1 PVNPs were determined by EIA-based glycan binding assays. The results showed that the PVNPs bound only 2,3-linked, but not 2,6- or 2,8-linked sialoglycans (FIG. 6(b)), consistent with the known receptor binding nature of their parental H7N9 avian IVs [35, 37, 38]. Accordingly, the S60-HA1 PVNPs agglutinated human RBCs at a high hemagglutination titer of 122 ng/mL (FIG. 6(c)). As negative controls, the S60 nanoparticles did not bind any tested sialoglycans and did not cause hemagglutination. These data collectively demonstrated that the PVNP-displayed HA1 antigens retain the authentic structure, conformation, receptor binding function, and hemagglutination nature, supporting that the S60-HA1 PVNPs may serve as a reagent for IV study.

### 3.7 High Immune Response of the S60-HA1 PVNPs

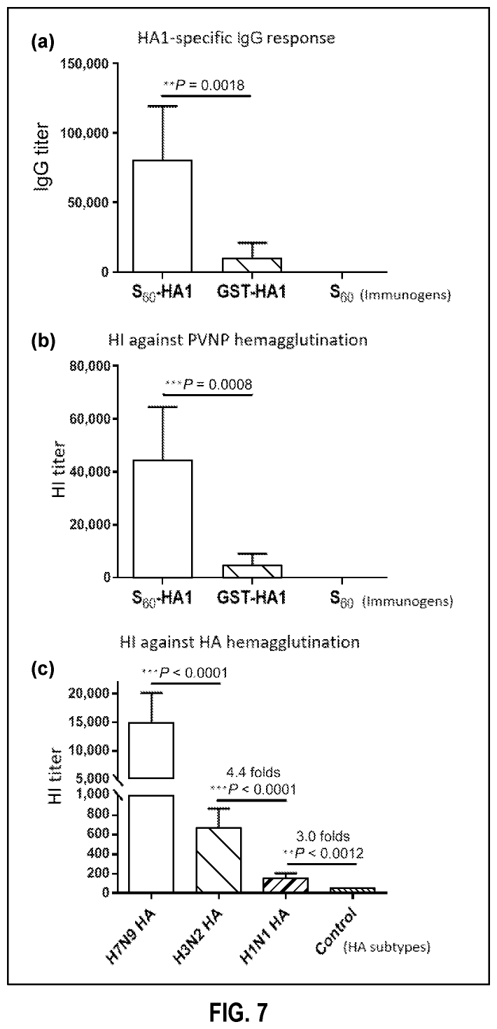

Numerous literatures show that antigens with pathogen associated molecular patterns (PAMPs) in polyvalent nature induce stronger immune responses compared with the mono- or low-valent ones [39-42]. To prove that this is true for the PVNPs, mice were immunized with the S60-HA1 PVNPs using dimeric GST-HA1 proteins for comparison. After three immunizations, mouse sera were collected to determine HA1-specific IgG titers using recombinant H7 HA1 protein (Sino Biological) as capture antigen. The outcomes showed that the PVNP-elicited HA1-specific IgG titer (80,000) was 8-fold higher than that (10,000) induced by the GST-HA1 protein (P=0.0018, FIG. 7(a)), supporting the above notion.

### 3.8 High Hemagglutination Inhibition (HI) Effects of the PVNP Immunized Mouse Sera

Applicant then measured the inhibition effects of the mouse sera against hemagglutination of the S60-HA1 PVNPs, revealing high HI titers (44,373) of the PVNP-immunized sera that was about 9.5 folds greater than that (4,693) of the mouse sera after immunization with the GST-HA1 protein (P=0.0008, FIG. 7(b)), consistent with their IgG titers. Applicant determined HI titers of the sera against hemagglutination of various commercial recombinant HA proteins (Sino Biological). The results showed that the S60-HA1 PVNP-immunized mouse sera showed very high HI titer (14,933) against hemagglutination of the homologous H7 (A/Zhejiang/1/2013/H7N9) HA protein (FIG. 7(c)). Noteworthy, the sera also showed high HI titer (667) to heterologous subtype H3 (A/California/7/2004/H3N2) HA protein that clusters with the H7 subtype in the IAV group 2, but relatively low HI titer (150) to heterologous subtype H1 (A/NewYork/18/2009/H1N1) HA proteins that belongs to the IAV group 1 (FIG. 7(c)). As negative controls, the S60 nanoparticle did not elicit HA1-specific antibody (FIG. 7(a)) and the resulted mouse sera did not inhibit the hemagglutinations by the S60-HA1 H7 PVNPs (FIG. 7(c), control) or other tested recombinant HA proteins (data not shown). As such, the data support the use of the S60-HA1 PVNPs as a flu vaccine candidate.

### 4 Discussion

In this study Applicant designed and generated the S60-HA1 PVNPs by taking advantage of the self-formation of the S60 nanoparticles to display the key neutralizing HA1 antigens of IVs for enhanced immunogenicity. The self-assembled S60-HA1 PVNPs can be produced through the prokaryotic E. coli system, as well as the eukaryotic mammalian cell system. The bacterially expressed PVNPs have been well characterized structurally and functionally in this study. Each PVNP has an S60 nanoparticle core that resembles the inner shell of NoV capsid and 60 surface-displayed HA1 antigens extending from the inner shell, making the PVNPs resembling virus-like particles. Remarkably, the exposed HA1 antigens are arranged into trimers, like those on the HAs of authentic flu viruses. Functional data showed that the PVNPs were recognized by HA-specific antibody, bound specific sialic acid receptors, and agglutinated human RBCs. These outcomes indicated that the PVNP displayed HA1 antigens retain the original structures, conformational epitopes, receptor binding functions, and PAMPs. Thus, the S60-HA1 PVNPs may be used as reagents to study influenza viruses. As a result of polyvalent presentations of the HA1 antigens by the PVNPs, and the well-preserved PAMPs of both HA1 antigens and NoV S60-nanoparticles, the S60-HA1 PVNPs elicited strong immune responses. These were shown by the high HA1-specific IgG titers in the PVNP immunized mouse sera, as well as their high HI titers against hemagglutination of the S60-HA1 PVNPs and various recombinant HA proteins. The mouse sera after immunization with the S60-HA1 PVNPs exhibited very high HI titers, reaching to 14,933, against hemagglutination of the homologous H7 HA proteins. This strongly suggested that the S60-HA1 PVNPs may serve as a potent vaccine candidate against H7 IAVs, such as the highly pathogenic H7N9 avian IAVs that caused five epidemic waves from 2013 to 2017 in China, claiming 511 human lives [43]. The high HI titer resulted apparently from the specific binding of the serum antibody to the HA1 antigens of the H7 HA proteins and thus inhibited the hemagglutination ability of the HA protein. Noteworthy, the S60-HA1 PVNP-immunized mouse sera also inhibited hemagglutination of heterologous H3 (A/California/7/2004/H3N2) HA protein with a high HI titer of 667, which was significantly higher than that (150) against H1 (A/New York/18/2009/H1N1) HA proteins (P<0.0001). These data implied certain cross protection of future S60-HA1 H7 PVNP vaccine against the two heterologous IAV subtypes. The higher HI titers of the H7 PVNP-immunized sera against the H3 HA protein than that of the H1 HA protein may be explained by the facts that H7 and H3 subtypes are genetically closer related, clustering in the same IAV group 2, while H1 belongs to IAV group 1, being genetically further away from the H7 subtype.

Influenza virus HA1 antigens are known for their variable sequences, leading to antigenic drifts and epochal evolution of flu viruses. However, a conserved antigenic supersite has been reported at or near the receptor binding site (RBS) that is targeted by broadly neutralizing antibodies [44-46]. Similarly, by analyzing the HA1 sequences of major H1 IVs circulated over the past century, a naturally protective epitope of limited variability near the RBS has been identified and proposed as a target of a universal flu vaccine [47]. Displaying the HA1 domain alone by the polyvalent S60 nanoparticle may elicit substantially high proportion of antibody specific to the antigenic supersite and/or the conserved protective epitope. This may explain the observed high cross subtype HI titers. In fact, a previous study showed that bacterially expressed, oligomerized HA1 s elicited cross-clade protective immunity in ferrets [48]. Thus, while a PVNP cocktail with multiple HA1 antigens of predominant IVs may offer broad protection (see above), alternatively, a pool of PVNPs that display a collection of native and/or artificially designed protective epitopes of limited variability may be generated for broad protection against designated IVs. Finally, to the question that the PVNP containing only HA1s without other IV antigens might not elicit complete protective immunity as the primary vaccination, such PVNPs may further be useful as a boosting vaccine, because the vast majority of human adults have been infected and/or immunized with one or more IVs and/or flu vaccines and thus have already memories to the conserved IV epitopes. In this case, the immunity against the variable HA1 antigens of currently circulated IVs may be the key to confer protection against the new IV strains.

Applicant noted that the PVNPs formed by the S-HA1 proteins were heterologous in sizes. In vitro expression of NoV VP1s often leads to VLP formation at heterologous sizes, including those in T=1 (˜31 nm), 3 (˜46 nm), and 4 (˜50 nm) icosahedral symmetry, consisting of 60, 180, and 240 NoV VP1s, respectively [49, 50]. By focusing on the major populations of the His-tagged H7 PVNPs, Applicant reconstructed the 3D structures of two PVNPs with distinct diameters (21 nm vs. 16 nm) by cryoEM method, revealing the same T=1 icosahedral symmetry, being composed of 60 copies of the S-HA1 fusion proteins. This size difference appears to be resulted from their different ways of homotypic interactions among the S-HA1 fusion proteins during PVNP assembly. Even larger PVNPs observed in this study may represent those in T=3 icosahedral symmetry.

Despite size variations, the PVNPs should share the basic organization comprising of the icosahedral inner shell formed by the NoV S domains and the surface displayed HA1 antigens. As explained above, the polyvalent PVNPs and their well-preserved PAMPs enhance the immunogenicity of the HA1 antigens, leading to the high immune responses towards the HA1 antigens. Thus, the size variations should not affect the potential of the PVNPs as an excellent IV immunogen. Such size heterogeneity of PVNPs may be reduced by using a mammalian cell expression system that is known to provide better folding environments for viral structural proteins. HA1 antigens may be glycosylated, the mammalian cell system a better providing a chance to reconstruct their original glycosylation and other post translational modifications, making the resulting PVNPs a better flu vaccine candidate. On the other hand, the above explained protective epitope of limited variability near the RBS has been shown to be a peptide epitope without glycosylation [47]. This justifies the observed high HI effects of the PVNP immunized sera in this study, supporting the use of the PVNPs as a useful flu vaccine.

The readily available PVNPs displaying the HA1 antigens of H7 subtype, as well as similar PVNPs having other HA1 antigens, may be generated using the disclosed procedures to offer convenient and useful reagents to study flu viruses. These PVNPs can be produced either via the prokaryotic E. coli system quickly at a low cost or through a eukaryotic system potentially for better structural and functional integrity of the HA1 antigens. During this study, the PVNPs remained structurally intact after storage at −80 and −20° C. for at least a year, as well as 4° C. for at least six months. In addition, the PVNPs were shown to retain their structural integrity after lyophilization and rehydration treatments, offering a further useful approach for PVNP preservation. The PVNPs with polyvalent HA1 antigens will be able to better mimic the HA features of flu viruses, providing stronger antigenic reactivity, greater receptor binding avidity, and higher immune responses, than those of commercially available recombinant monomeric or trimeric HA proteins.

### REFERENCES

[1] WHO. Seasonal influenza represents a year-round disease burden [Online]. www.who.int/news-room/feature-stories/detail/8-things-to-know-about-pandemic-influenza. Fischer II, W. A.; Gong, M.; Bhagwanjee, S.; Sevransky, J. Global burden of influenza as a cause of cardiopulmonary morbidity and mortality. Glob Heart 2014, 9, 325-336.
[2] Iuliano, A. D.; Roguski, K. M.; Chang, H. H.; Muscatello, D. J.; Palekar, R.; Tempia, S.; Cohen, C.; Gran, J. M.; Schanzer, D.; Cowling, B. J. et al. Estimates of global seasonal influenzaassociated respiratory mortality: A modelling study. Lancet 2018, 391, 1285-1300.
[3] Centers for Disease Control and Prevention. Seasonal influenza vaccine effectiveness, 2005-2020 [Online]. https://www.cdc.gov/flu/vaccines-work/effectiveness-studies.htm (accessed 2020).
[4] Wu, N. C.; Zost, S. J.; Thompson, A. J.; Oyen, D.; Nycholat, C. M.; McBride, R.; Paulson, J. C.; Hensley, S. E.; Wilson, I. A. A structural explanation for the low effectiveness of the seasonal influenza H3N2 vaccine. PLoS Pathog 2017, 13, e1006682.
[5] Skowronski, D. M.; Janjua, N. Z.; De Serres, G.; Sabaiduc, S.; Eshaghi, A.; Dickinson, J. A.; Fonseca, K.; Winter, A. L.; Gubbay, J. B.; Krajden, M. et al. Low 2012-13 influenza vaccine effectiveness associated with mutation in the egg-adapted H3N2 vaccine strain not antigenic drift in circulating viruses. PLoS One 2014, 9, e92153.
[6] Paules, C. I.; Sullivan, S. G.; Subbarao, K.; Fauci, A. S. Chasing seasonal influenza—the need for a universal influenza vaccine. New Engl. J. Med. 2018, 378, 7-9.
[7] Gouma, S.; Zost, S. J.; Parkhouse, K.; Branche, A.; Topham, D. J.; Cobey, S.; Hensley, S. E. Comparison of human H3N2 antibody responses elicited by egg-based, cell-based, and recombinant protein based influenza vaccines during the 2017-2018 season. Clin. Infect. Dis. 2020, 71, 1447-1453.
[8] Zost, S. J.; Parkhouse, K.; Gumina, M. E.; Kim, K.; Perez, S. D.; Wilson, P. C.; Treanor, J. J.; Sant, A. J.; Cobey, S.; Hensley, S. E. Contemporary H3N2 influenza viruses have a glycosylation site that alters binding of antibodies elicited by egg-adapted vaccine strains. Proc. Natl. Acad. Sci. USA 2017, 114, 12578-12583.
[9] Levine, M. Z.; Martin, E. T.; Petrie, J. G.; Lauring, A. S.; Holiday, C.; Jefferson, S.; Fitzsimmons, W. J.; Johnson, E.; Ferdinands, J. M.; Monto, A. S. Antibodies against egg- and cell-grown influenza A(H3N2) viruses in adults hospitalized during the 2017-2018 influenza season. J. Infect. Dis. 2019, 219, 1904-1912.
[10] Rolfes, M. A.; Flannery, B.; Chung, J. R.; O'Halloran, A.; Garg, S.; Belongia, E. A.; Gaglani, M.; Zimmerman, R. K.; Jackson, M. L.; Monto, A. S. et al. Effects of influenza vaccination in the United States during the 2017-2018 influenza season. Clin. Infect. Dis. 2019, 69, 1845-1853.
[11] Kanekiyo, M.; Wei, C. J.; Yassine, H. M.; McTamney, P. M.; Boyington, J. C.; Whittle, J. R. R.; Rao, S. S.; Kong, W. P.; Wang, L. S.; Nabel, G. J. Self-assembling influenza nanoparticle vaccines elicit broadly neutralizing H1N1 antibodies. Nature 2013, 499, 102-106.
[12] Yassine, H. M.; Boyington, J. C.; McTamney, P. M.; Wei, C. J.; Kanekiyo, M.; Kong, W. P.; Gallagher, J. R.; Wang, L. S.; Zhang, Y.; Joyce, M. G. et al. Hemagglutinin-stem nanoparticles generate heterosubtypic influenza protection. Nat. Med. 2015, 21, 1065-1070.
[13] Kanekiyo, M.; Joyce, M. G.; Gillespie, R. A.; Gallagher, J. R.; Andrews, S. F.; Yassine, H. M.; Wheatley, A. K.; Fisher, B. E.; Ambrozak, D. R.; Creanga, A. et al. Mosaic nanoparticle display of diverse influenza virus hemagglutinins elicits broad B cell responses. Nat. Immunol. 2019, 20, 362-372.
[14] Boyoglu-Barnum, S.; Ellis, D.; Gillespie, R. A.; Hutchinson, G. B.; Park, Y. J.; Moin, S. M.; Acton, O.; Ravichandran, R.; Murphy, M.; Pettie, D. et al. Elicitation of broadly protective immunity to influenza by multivalent hemagglutinin nanoparticle vaccines. bioRxiv 2020.05.30.125179, 2020. Available at: https://doi.org/10.1101/2020.05.30.125179.
[15] Bale, J. B.; Gonen, S.; Liu, Y. X.; Sheffler, W.; Ellis, D.; Thomas, C.; Cascio, D.; Yeates, T. O.; Gonen, T.; King, N. P. et al. Accurate design of megadalton-scale two-component icosahedral protein complexes. Science 2016, 353, 389-394.
[16] D'Aoust, M. A.; Couture, M. M. J.; Charland, N.; Trepanier, S.; Landry, N.; Ors, F.; Vézina, L. P. The production of hemagglutinin based virus-like particles in plants: A rapid, efficient and safe response to pandemic influenza. Plant Biotechnol. J. 2010, 8, 607-619.
[17] Smith, G.; Liu, Y.; Flyer, D.; Massare, M. J.; Zhou, B.; Patel, N.; Ellingsworth, L.; Lewis, M.; Cummings, J. F.; Glenn, G. Novel hemagglutinin nanoparticle influenza vaccine with Matrix-MTM adjuvant induces hemagglutination inhibition, neutralizing, and protective responses in ferrets against homologous and drifted A(H3N2) subtypes. Vaccine 2017, 35, 5366-5372.
[18] Portnoff, A. D.; Patel, N.; Massare, M. J.; Zhou, H. X.; Tian, J. H.; Zhou, B.; Shinde, V.; Glenn, G. M.; Smith, G. Influenza hemagglutinin nanoparticle vaccine elicits broadly neutralizing antibodies against structurally distinct domains of H3N2 HA. Vaccines 2020, 8, 99.
[19] Xia, M.; Huang, P. W.; Sun, C.; Han, L.; Vago, F. S.; Li, K. P.; Zhong, W. M.; Jiang, W.; Klassen, J. S.; Jiang, X. et al. Bioengineered norovirus S60 nanoparticles as a multifunctional vaccine platform. ACS Nano 2018, 12, 10665-10682.
[20] Xia, M.; Huang, P. W.; Jiang, X.; Tan, M Immune response and protective efficacy of the S particle presented rotavirus VP8* vaccine in mice. Vaccine 2019, 37, 4103-4110.
[21] Tan, M.; Cui, L. B.; Huo, X.; Xia, M.; Shi, F. J.; Zeng, X. Y.; Huang, P. W.; Zhong, W. M.; Li, W. W.; Xu, K. et al. Saliva as a source of reagent to study human susceptibility to avian influenza H7N9 virus infection. Emerg. Microbes Infect. 2018, 7, 1-10.
[22] Tan, M.; Jiang, X. The P domain of norovirus capsid protein forms a subviral particle that binds to histo-blood group antigen receptors. J. Virol. 2005, 79, 14017-14030.
[23] Tan, M.; Hegde, R. S.; Jiang, X. The P domain of norovirus capsid protein forms dimer and binds to histo-blood group antigen receptors. J. Virol. 2004, 78, 6233-6242.
[24] Xia, M.; Huang, P. W.; Jiang, X.; Tan, M. A nanoparticle-based trivalent vaccine targeting the glycan binding VP8* domains of rotaviruses. Viruses 2021, 13, 72.
[25] Tan, M.; Huang, P. W.; Xia, M.; Fang, P. A.; Zhong, W. M.; McNeal, M.; Wei, C.; Jiang, W.; Jiang, X. Norovirus P particle, a novel platform for vaccine development and antibody production. J. Virol. 2011, 85, 753-764.
[26] Wang, L. Y.; Huang, P. W.; Fang, H.; Xia, M.; Zhong, W. M.; McNeal, M. M.; Jiang, X.; Tan, M. Polyvalent complexes for vaccine development. Biomaterials 2013, 34, 4480-4492.
[27] Xia, M.; Wei, C.; Wang, L. Y.; Cao, D. J.; Meng, X. J.; Jiang, X.; Tan, M. Development and evaluation of two subunit vaccine candidates containing antigens of hepatitis E virus, rotavirus, and astrovirus. Sci. Rep. 2016, 6, 25735.
[28] Xia, M.; Wei, C.; Wang, L. Y.; Cao, D. J.; Meng, X. J.; Jiang, X.; Tan, M. A trivalent vaccine candidate against hepatitis E virus, norovirus, and astrovirus. Vaccine 2016, 34, 905-913.
[29] Huang, P. W.; Farkas, T.; Zhong, W. M.; Tan, M.; Thornton, S.; Morrow, A. L.; Jiang, X. Norovirus and histo-blood group antigens: Demonstration of a wide spectrum of strain specificities and classification of two major binding groups among multiple binding patterns. J. Virol. 2005, 79, 6714-6722.
[30] Tan, M.; Fang, P. A.; Xia, M.; Chachiyo, T.; Jiang, W.; Jiang, X. Terminal modifications of norovirus P domain resulted in a new type of subviral particles, the small P particles. Virology 2011, 410, 345-352.
[31] Tan, M.; Fang, P. A.; Chachiyo, T.; Xia, M.; Huang, P. W.; Fang, Z. Y.; Jiang, W.; Jiang, X. Noroviral P particle: Structure, function and applications in virus-host interaction. Virology 2008, 382, 115-123.
[32] Punjani, A.; Rubinstein, J. L.; Fleet, D. J.; Brubaker, M. A. cryoSPARC: Algorithms for rapid unsupervised cryo-EM structure determination. Nat. Methods 2017, 14, 290-296.
[33] Punjani, A.; Rubinstein, J. L.; Fleet, D. J.; Brubaker, M. A. cryoSPARC: Algorithms for rapid unsupervised cryo-EM structure determination. Nat. Methods 2017, 14, 290-296
[34] Burmeister, W. P.; Buisson, M.; Estrozi, L. F.; Schoehn, G.; Billet, O.; Hannas, Z.; Sigoillot, C.; Poulet, H. Structure determination of feline calicivirus virus-like particles in the context of a pseudooctahedral arrangement. PLoS One 2015, 10, e0119289. Shi, Y.; Zhang, W.; Wang, F.; Qi, J. X.; Wu, Y.; Song, H.; Gao, F.; Bi, Y. H.; Zhang, Y. F.; Fan, Z. et al. Structures and receptor binding of hemagglutinins from human-infecting H7N9 influenza viruses. Science 2013, 342, 243-247.
[35] Yang, H.; Carney, P. J.; Chang, J. C.; Villanueva, J. M.; Stevens, J. Structural analysis of the hemagglutinin from the recent 2013 H7N9 influenza virus. J. Virol. 2013, 87, 12433-12446.
[36] Xu, R.; De Vries, R. P.; Zhu, X. Y.; Nycholat, C. M.; McBride, R.; Yu, W. L.; Paulson, J. C.; Wilson, I. A. Preferential recognition of avian-like receptors in human influenza A H7N9 viruses. Science 2013, 342, 1230-1235.
[37] Tharakaraman, K.; Jayaraman, A.; Raman, R.; Viswanathan, K.; Stebbins, N. W.; Johnson, D.; Shriver, Z.; Sasisekharan, V.; Sasisekharan, R. Glycan receptor binding of the influenza A virus H7N9 hemagglutinin. Cell 2013, 153, 1486-1493.
[38] Tan, M.; Jiang, X. Norovirus capsid protein-derived nanoparticles and polymers as versatile platforms for antigen presentation and vaccine development. Pharmaceutics 2019, 11, 472.
[39] Tan, M.; Jiang, X. Recent advancements in combination subunit vaccine development. Hum. Vaccin. Immunother. 2017, 13, 180-185.
[40] Khudyakov, Y.; Pumpens, P. Nanoparticles of norovirus. In Viral Nanotechnology. Khudyakov, Y.; Pumpens, P., Eds.; CRC Press: Boca Raton, 2015; pp 363-371.
[41] Tan, M.; Jiang, X. Subviral particle as vaccine and vaccine platform. Curr. Opin. Virol. 2014, 6, 24-33.
[42] Su, S.; Gu, M.; Liu, D.; Cui, J.; Gao, G. F.; Zhou, J. Y.; Liu, X. F. Epidemiology, evolution, and pathogenesis of H7N9 influenza viruses in five epidemic waves since 2013 in China. Trends Microbiol. 2017, 25, 713-728.
[43] Ekiert, D. C.; Kashyap, A. K.; Steel, J.; Rubrum, A.; Bhabha, G.; Khayat, R.; Lee, J. H.; Dillon, M. A.; O'Neil, R. E.; Faynboym, A. M. et al. Cross-neutralization of influenza A viruses mediated by a single antibody loop. Nature 2012, 489, 526-532.
[44] Whittle, J. R. R.; Zhang, R. J.; Khurana, S.; King, L. R.; Manischewitz, J.; Golding, H.; Dormitzer, P. R.; Haynes, B. F.; Walter, E. B.; Moody, M. A. et al. Broadly neutralizing human antibody that recognizes the receptor-binding pocket of influenza virus hemagglutinin. Proc. Natl. Acad. Sci. USA 2011, 108, 14216-14221.
[45] Lee, P. S.; Ohshima, N.; Stanfield, R. L.; Yu, W. L.; Iba, Y.; Okuno, Y.; Kurosawa, Y.; Wilson, I. A. Receptor mimicry by antibody F045-092 facilitates universal binding to the H3 subtype of influenza virus. Nat. Commun. 2014, 5, 3614.
[46] Thompson, C. P.; Lourenco, J.; Walters, A. A.; Obolski, U.; Edmans, M.; Palmer, D. S.; Kooblall, K.; Carnell, G. W.; O'Connor, D.; Bowden, T. A. et al. A naturally protective epitope of limited variability as an influenza vaccine target. Nat. Commun. 2018, 9, 3859.
[47] Khurana, S.; Verma, S.; Verma, N.; Crevar, C. J.; Carter, D. M.; Manischewitz, J.; King, L. R.; Ross, T. M.; Golding, H. Bacterial HA1 vaccine against pandemic H5N1 influenza virus: Evidence of oligomerization, hemagglutination, and cross-protective immunity in ferrets. J. Virol. 2011, 85, 1246-1256.
[48] Jung, J.; Grant, T.; Thomas, D. R.; Diehnelt, C. W.; Grigorieff, N.; Joshua-Tor, L. High-resolution cryo-EM structures of outbreak strain human norovirus shells reveal size variations. Proc. Natl. Acad. Sci. USA 2019, 116, 12828-12832.
[49] Devant, J. M.; Hansman, G. S. Structural heterogeneity of a human norovirus vaccine candidate. Virology 2021, 553, 23-34.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A pseudovirus nanoparticle (PVNP) composition or consisting of an aggregate of fusion proteins forming an icosahedral structure and a nanoparticle shell, said fusion protein comprising
a) a modified norovirus (NoV) S domain protein;
b) a hemagglutinin I (HA1) antigen of the influenza hemagglutinin I (HA1) of influenza virus; and
c) a peptide linker, said peptide linker connecting the C-terminus of said NoV S domain to said HA1 antigen;
wherein said modified NoV S domain protein forms the interior nanoparticle shell of said PVNP composition;
wherein said inner nanoparticle shell displays 60 exposed C-termini of said S domain in a triangular pattern;
wherein 60 HA1 antigens are displayed on the surface of said nanoparticle shell;
wherein said 60 HA1 antigens form 20 HA1 trimers.

2. The PVNP composition of claim 1, wherein said PVNP has a diameter of about 21 nanometers.

3. The PVNP composition of claim 1, wherein said NoV S domain comprises or consists of at least 90%, or at least 91%, or at least 92% or at least 93%, or at least 94%, or at least 95% or at least 96%, or at least 97%, or at least 98%, or at least 99%, or 100% homology to the sequence
(SEQ ID NO: 1)
MKMASNDASPSDGSTANLVPEVNNEVMALEPVVGAAIAAPVAGQQNVID
PWIRNNFVQAPGGEFTVSPANAPGEILWSAPLGPDLNPYLSHLARMYNG
YAGGFEVQVILAGNAFTAGKVIFAAVPPNFPTEGLSPSQVTMFPHIIVD
VRQLEPVLIPLPDVRNNFYHYNQSNDSTIKLIAMLYTPLRANNAGDDVF
TVS.

4. The PVNP composition of any preceding claim, wherein said NoV S domain sequence is wildtype at one or more of positions V57, Q58, or S136, and M140.

5. The PVNP composition of any preceding claim, wherein said NoV S domain sequence is wildtype at all positions V57, Q58, or 5136, and M140.

6. The PVNP of any preceding claim, wherein said protein sequence encoding a norovirus (NoV) S domain is wild-type at positions 57, 58, 136, and 140.

7. The PVNP composition of any preceding claim, wherein said HA1 antigen is of an H7N9 influenza virus subtype.

8. The PVNP composition of any preceding claim, wherein said HA1 antigen is of an H7N9 influenza virus subtype and has at least 90% sequence homology to SEQ ID NO: 2.

9. The PVNP composition of any of claims 1 through 8, wherein said HA1 antigen is of an H1N1 subtype.

10. The PVNP composition of any preceding claim, wherein said HA1 antigen is of an H1N1 influenza virus subtype and has at least 90% sequence homology to SEQ ID NO: 3.

11. The PVNP composition of any of claims 1 through 8, wherein said HA1 antigen is of an H3N2 subtype.

12. The PVNP composition of any of claims 1 through 8, wherein said HA1 antigen is of an H3N2 influenza virus subtype and has at least 90% sequence homology to SEQ ID NO: 4.

13. The PVNP composition of any of claims 1 through 8, wherein said HA1 antigen is of an influenza B virus subtype.

14. The PVNP composition of any of claims 1 through 8, wherein said HA1 antigen is of an influenza B virus subtype and has at least 90% sequence homology to SEQ ID NO: 5.

15. The PVNP composition of any preceding claim, wherein said HA1 antigen comprises or consists of a region covering the receptor binding site (RBS).

16. The PVNP composition of any preceding claim wherein said HA1 antigen is glycosylated.

17. The PVNP composition of any preceding claim, said linker comprises or consists of a sequence selected from HHHH (SEQ ID NO: 6), GGGG (SEQ ID NO:7), and GSGS (SEQ ID NO:8).

18. The PVNP composition of any preceding claim, wherein said fusion protein is tag-free.

19. The PVNP composition of any preceding claim, wherein said PVNP has T=1 icosahedral symmetry.

20. The PVNP composition of any preceding claim, wherein said PVNP induces substantial immunity to influenza virus infection or at least one symptom thereof when administered to a subject.

21. A method of immunizing an individual against one or both of an H7 IAV or H3N2 or H7N9 avian IAV, comprising administering the composition of any preceding claim.

22. A method of eliciting an immune response in a subject against an influenza virus in an individual in need thereof against an influenza virus, comprising administering a composition of any preceding claim, in an amount effective to produce an antigen-specific immune response in said individual.

23. The method of claim 22, wherein said composition is administered as a dose.

24. The method of claim 21 or 22, wherein said composition is administered to an individual via a route selected from one or more of intradermal injection, intramuscular injection, or by intranasal administration.

25. The method of any of claims 22 through 24, said composition comprising a pharmaceutically acceptable carrier.

26. The method of any of claims 22 through 25, said composition comprising one or more of an adjuvant and a preservative.

27. The method of any of claims 23 through 26, said dose comprising from about 1 to about 150 ug of PVNP.

28. The method of any of claims 22 through 27, wherein said composition is sterile.

29. A method of making a pseudovirus nanoparticle (PVNP), comprising
a. providing a plasmid comprising gene sequences corresponding to a modified NoV S domain, a linker, and an HA1 antigen;
b. expressing a gene product of said gene sequences in an E. coli or a eukaryotic cell system;
c. lysing the cells of said cell system expressing the S-HA1 protein; and
d. precipitating said gene product from said lysed cell system with (NH₄)₂SO₄;
wherein said sequence is tag-free; and
wherein said concentration of (NH₄)₂SO₄ is between about 0.7 and about 1.2 M.

30. The method of claim 29, wherein said cell system is selected from a yeast system, a baculovirus/insect cell system, and a mammalian cell system.

31. A reagent for the study of an influenza virus, comprising the composition of any of claims 1 through 19.

32. The reagent of claim 31, said reagent comprising a PVNP capable of remaining structurally intact after storage at −80° C. and −20° C. for at least a year, and at 4° C. for at least six months.

33. The reagent of claim 31, said reagent comprising a PVNP capable of retaining structural integrity after lyophilization and rehydration treatment.

34. The reagent of claim 31, said reagent comprising a PVNP having a polyvalent HA1 antigen, said polyvalent HA1 antigen having one or more of antigenic reactivity, receptor binding, and ability to cause an immune response.

35. A reagent for the study of an influenza virus, comprising a prokaryotic cell system and a vector expressing one or more of the PVNP of any of claims 1 through 19.

36. The reagent of claim 34, said prokaryotic cell system being an E. coli system.

37. A pseudovirus nanoparticle (PVNP), said PVNP comprising a plurality of fusion proteins, said fusion protein comprising a modified norovirus (NoV) S domain protein, a hemagglutinin I (HA1) antigen of the influenza hemagglutinin I (HA1) of an influenza virus, said S domain protein and HA1 antigen being connected via a peptide linker;
wherein said plurality of said fusion proteins forms a nanoparticle having an icosahedral structure,
wherein said nanoparticle has 60 HA1 antigens presented at the surface of said nanoparticle, said 60 HA1 antigens arranged to form 20 HA trimers;
wherein said HA1 antigen has a sequence length of from about 225 to about 240 amino acids, and from about 95% to 100% sequence homology to a sequence selected from SEQ NO: 2, SEQ NO: 3, SEQ NO: 4 or SEQ NO: 5.
